(19) United States
(12) Patent Application Publication
(10) Pub. No.: US 2025/0136983 A1
(43) Pub. Date: May 1, 2025

(54) Composition and Methods for Modulating Nuclear Enriched Abundant Transcript 1 to Treat Cognitive Impairment

(71) Applicant: MUSC Foundation for Research Development, Charleston, SC (US)

(72) Inventor: Hongkuan Fan, Mt Pleasant, SC (US)

(21) Appl. No.: 18/835,880

(22) PCT Filed: Feb. 10, 2023
(86) PCT No.: PCT/US2023/062360

§ 371 Date: Aug. 5, 2024

Related U.S. Application Data
(60) Provisional application No. 63/309,190, filed on Feb. 11, 2022.

(51) Int. Cl. C12N 15/113; A61P 25/28

(52) U.S. Cl. CPC C12N 15/113; A61P 25/28; C12N 2310/113; C12N 2310/315; C12N 2310/3231; C12N 2310/341

(57) ABSTRACT

The invention provides compositions for modulating the level of Neat1 lncRNA and methods of using the compositions for treating neurodegenerative diseases or disorders.

### CROSS REFERENCE TO RELATED APPLICATIONS

This application claims priority to U.S. Provisional Application No. 63/309,190, filed Feb. 11, 2022 which is hereby incorporated by reference herein in its entirety.

### STATEMENT REGARDING FEDERALLY SPONSORED RESEARCH OR DEVELOPMENT

This invention was made with government support under R01GM130653 awarded by the National Institutes of Health. The government has certain rights in the invention.

### BACKGROUND OF THE INVENTION

Although sepsis incidence has steadily increased in recent years, sepsis morality rates have declined leading to an expanding population of sepsis survivors (Rhee et al., 2020, J Thorac Dis 12, S89-S100; Kaukonen et al., 2014, JAMA 311, 1308-1316; Iwashyna et al., 2012, J Am Geriatr Soc 60, 1070-1077). This population frequently suffers from sepsis-associated encephalopathy (SAE) which has been associated with long-term functional sequelae including cognitive impairment, anxiety, depression, and post-traumatic stress disorder (PTSD) (Pandharipande et al., 2014, N Engl J Med 370, 185-186; Rahmel et al., 2020, PLoS One 15, e0228952). This so-called ‘Post-ICU syndrome’ contributes to the excess mortality that occurs within five years after sepsis (Annane et al., 2015, Lancet Respir Med 3, 61-69). The pathogenesis of sepsis-induced cognitive impairment is poorly understood although growing evidence indicates that neuroinflammation and related excitotoxicity may play a role (Stubbs et al., 2013, Nat Rev Neurol 9, 551-561; Zhou et al., 2020, CNS Neurosci Ther 26, 177-188).

Neuron synapse loss correlates with the cognitive deficit and synaptic function is regulated by altering the expression level of synaptic scaffold proteins such as postsynaptic density protein 95 (PSD-95)—a member of the membrane-associated guanylate kinase (MAGUK) class of proteins at synapses (El-Husseini et al., 2000, Science 290, 1364-1368; Diering et al., 2018, Neuron 100, 314-329). PSD-95 is an essential component involved in synaptic plasticity and dendritic spine morphogenesis during neurodevelopment (Gilman et al., 2011, Neuron 70, 898-907) and several genomic studies link PSD-95 dysfunctions to neuropsychiatric disorders (Fromer et al., 2014, Nature 506, 179-184; Fossati et al., 2015, Cell Death Differ 22, 1425-1436; Coley et al., 2019, Sci Rep 9, 9486). PSD-95 protein expression levels are downregulated in the brain after sepsis (Xiong et al., 2019, Inflammation 42, 354-364; Moraes et al., 2015, Mol Neurobiol 52, 653-663) and may represent an underexplored contributor to SAE pathogenesis. Evolving evidence has also demonstrated the potential importance of hemoglobin (Hb) in sepsis-related organ failure via cellular dissociation and resultant inflammatory modulation, nitric oxide scavenging and free radical injury (Shaver et al., 2019, Am J Physiol Renal Physiol 317, F922-F929; Meegan et al., 2020, PLoS One 15, e0228727; Janz et al., 2013, Crit Care Med 41, 784-790). Hemoglobin expression was previously felt to be specific to erythrocytes; however, it has more recently been observed in several cells including neurons and glial cells (Biagioli et al., 2009, Proc Natl Acad Sci USA 106, 15454-15459) raising the question of its potential role in SAE through local injury. However, little is known about the potential interaction of Hb and PSD-95 and their role in neuronal injury in sepsis.

Long non-coding RNAs (lncRNAs) are a diverse subset of transcripts longer than 200 nucleotides that do not encode for proteins (Beermann et al., 2016, Physiol Rev 96, 1297-1325). Increasing evidence suggests that lncRNAs regulate almost every cellular function through various molecular mechanisms, including competing with endogenous mRNAs over microRNA binding, scaffolding of RNA-protein structures, and epigenetic regulation (Marchese et al., 2017, Genome Biol 18, 206; Devall et al., 2016, Neurosci Lett 625, 47-55). Neat1, nuclear-enriched abundant transcript 1, belongs to the group of lncRNA exhibiting highly abundant gene expression in the brain (Nakagawa et al., 2011, J Cell Biol 193, 31-39). Two isoforms of Neat1 that share the same promoter are recognized but differ in 3′-ends and length (3.7 and 23 kb in human and 3.2/20 kb in mice) (Yamazaki et al., 2015, Front Biosci (Elite Ed) 7, 1-41). Neat1 functions as a core scaffold of nuclear body paraspeckles that regulates transcription (Anantharaman et al., 2016, Sci Rep 6, 34043), which has also been proposed to control stress responses (Hirose et al., 2014, Mol Biol Cell 25, 169-183), activation of innate immune responses (Zhang et al., 2019, Nat Commun 10, 1495), and cellular differentiation (Shui et al., 2019, J Cell Physiol 234, 22477-22484) by sequestering RNA- and DNA-binding proteins (Hirose et al., 2014, Mol Biol Cell 25, 169-183), thus altering the epigenetic landscape of target gene promoters in favor of transcription (West et al., 2014, Mol Cell 55, 791-802). Altered Neat1 expression has been reported in many major neurodegenerative and psychiatric diseases, including frontotemporal dementia (FTD), Alzheimer's, Huntington's and Parkinson's diseases, amyotrophic lateral sclerosis (ALS), epilepsy, traumatic brain injury and schizophrenia (An et al., Noncoding RNA Res 3, 243-252; Butler et al., 2019, Sci Signal 12, (588):eaaw9277; Simchovitz et al., 2019, FASEB J 33, 11223-11234). Recently, several studies reported that Neat1 participated in sepsis-induced acute kidney injury (Chen et al., 2018, Int Immunopharmacol 59, 252-260), myocardial injury (Wang et al., 2019, Eur Rev Med Pharmacol Sci 23, 4898-4907) and liver injury (Zhang et al., 2019, Int Immunopharmacol 75, 105731). However, the role of Neat1 in SAE remains to be investigated.

There remains a need in the art for improved compositions and methods for treatment of neurodegenerative diseases. The present invention satisfies this unmet need.

### SUMMARY OF THE INVENTION

In one embodiment, the invention relates to a composition for treating or preventing a neurodegenerative disease or disorder, the composition comprising an inhibitor of the level or activity of at least one Neat1 lncRNA molecule, or a variant thereof. In one embodiment, the inhibitor is at least one selected from the group consisting of a chemical compound, a protein, a peptide, a peptidomemetic, a ribozyme, a small molecule chemical compound, a nucleic acid, a vector, an antibody, an antisense nucleic acid, a GapmeR, an siRNA, an shRNA, and a guide RNA.

In one embodiment, the composition comprises a GapmeR that targets Neat1 lncRNA. In one embodiment, the GapmeR comprises at least one locked nucleic acid (LNA). In one embodiment, the GapmeR comprises at least one LNA on each of the 5′ and 3′ end of the GapmeR sequence. In one embodiment, the GapmeR comprises at least one phosphorothioated LNA on each of the 5′ and 3′ end of the GapmeR sequence. In one embodiment, the composition comprises a GapmeR comprising a nucleotide sequence of SEQ ID NO:1, SEQ ID NO:2, SEQ ID NO:3, SEQ ID NO:4 or SEQ ID NO:5.

In one embodiment, the modulator inhibits the interaction of Neat1 and Hbb.

In one embodiment, the disease or disorder is septic induced cognitive impairment, COVID-19 induced cognitive impairment, sepsis-associated encephalopathy (SAE), mild cognitive impairment (MCI), frontotemporal dementia (FTD), epilepsy, traumatic brain injury, schizophrenia, polyQ disorders such as SCA1, SCA2, SCA3, SCA6, SCA7, SCA17, Huntington's disease, Dentatorubral-pallidoluysian atrophy (DRPLA), Alzheimer's disease, Parkinson's disease, amyotrophic lateral sclerosis (ALS), transmissible spongiform encephalopathies (prion disease), tauopathies, or Frontotemporal lobar degeneration (FTLD).

In one embodiment, the invention relates to a composition for inhibiting the level or activity of at least one Neat1 lncRNA molecule or a variant thereof.

In one embodiment, the inhibitor is a chemical compound, a protein, a peptide, a peptidomemetic, a ribozyme, a small molecule chemical compound, a nucleic acid, a vector, an antibody, an antisense nucleic acid, a GapmeR, an siRNA, an shRNA, or a guide RNA.

In one embodiment, the composition comprises a GapmeR that targets Neat1 lncRNA. In one embodiment, the GapmeR comprises at least one locked nucleic acid (LNA). In one embodiment, the GapmeR comprises at least one LNA on each of the 5′ and 3′ end of the GapmeR sequence. In one embodiment, the GapmeR comprises at least one phosphorothioated LNA on each of the 5′ and 3′ end of the GapmeR sequence. In one embodiment, the composition comprises a GapmeR comprising a nucleotide sequence of SEQ ID NO:1, SEQ ID NO:2, SEQ ID NO:3, SEQ ID NO:4 or SEQ ID NO:5.

In one embodiment, the inhibitor inhibits the interaction of Neat1 and Hbb.

In one embodiment, the invention relates to a method of treating or preventing a neurodegenerative disease or disorder in a subject in need thereof, the method comprising administering to the subject a composition comprising an inhibitor of the level or activity of at least one Neat1 lncRNA molecule, or a variant thereof. In one embodiment, the inhibitor is at least one selected from the group consisting of a chemical compound, a protein, a peptide, a peptidomemetic, a ribozyme, a small molecule chemical compound, a nucleic acid, a vector, an antibody, an antisense nucleic acid, a GapmeR, an siRNA, an shRNA, and a guide RNA.

In one embodiment, the composition comprises a GapmeR that targets Neat1 lncRNA. In one embodiment, the GapmeR comprises at least one locked nucleic acid (LNA). In one embodiment, the GapmeR comprises at least one LNA on each of the 5′ and 3′ end of the GapmeR sequence. In one embodiment, the GapmeR comprises at least one phosphorothioated LNA on each of the 5′ and 3′ end of the GapmeR sequence. In one embodiment, the composition comprises a GapmeR comprising a nucleotide sequence of SEQ ID NO:1, SEQ ID NO:2, SEQ ID NO:3, SEQ ID NO:4 or SEQ ID NO:5.

In one embodiment, the disease or disorder is septic induced cognitive impairment, sepsis-associated encephalopathy (SAE), COVID-19 induced cognitive impairment, mild cognitive impairment (MCI), frontotemporal dementia (FTD), epilepsy, traumatic brain injury, schizophrenia, polyQ disorders such as SCA1, SCA2, SCA3, SCA6, SCA7, SCA17, Huntington's disease, Dentatorubral-pallidoluysian atrophy (DRPLA), Alzheimer's disease, Parkinson's disease, amyotrophic lateral sclerosis (ALS), transmissible spongiform encephalopathies (prion disease), tauopathies, or Frontotemporal lobar degeneration (FTLD).

In one embodiment, the method of administration is intrathecal injection.

In one embodiment, the invention relates to a method of inhibiting Neat1 expression or activity, the method comprising administering to the subject a composition comprising an inhibitor a composition for inhibiting the level or activity of at least one Neat1 lncRNA molecule or a variant thereof. In one embodiment, the inhibitor is a chemical compound, a protein, a peptide, a peptidomemetic, a ribozyme, a small molecule chemical compound, a nucleic acid, a vector, an antibody, an antisense nucleic acid, a GapmeR, an siRNA, an shRNA, or a guide RNA. In one embodiment, the composition comprises a GapmeR that targets Neat1 lncRNA. In one embodiment, the GapmeR comprises at least one locked nucleic acid (LNA). In one embodiment, the GapmeR comprises at least one LNA on each of the 5′ and 3′ end of the GapmeR sequence. In one embodiment, the GapmeR comprises at least one phosphorothioated LNA on each of the 5′ and 3′ end of the GapmeR sequence. In one embodiment, the composition comprises a GapmeR comprising a nucleotide sequence of SEQ ID NO:1, SEQ ID NO:2, SEQ ID NO:3, SEQ ID NO:4 or SEQ ID NO:5.

### BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1A through FIG. 1F depict exemplary experimental data demonstrating that mice surviving CLP exhibit anxiety-like behavior, memory impairment and a decrease of dendritic spine density. FIG. 1A and FIG. 1B depict exemplary experimental data demonstrating that sham (n=23) and CLP (n=29) mice were subjected to open field test. FIG. 1A depicts representative tracks and heat map were shown. FIG. 1B depicts the frequency, time in the center zone and the first time for the mice to go to the center zone were analyzed (*p<0.05, **P<0.01). FIG. 1C depicts sham (n=12) and CLP (n=16) mice were subjected to contextual fear conditioning test and freezing behavior was analyzed (*P<0.05). FIG. 1D depicts differential expression of IEGs in neuronal cells between sham and CLP mice were analyzed at 24 h after surgery (*p<0.05, n=3). FIG. 1E depicts the PSD-95 protein levels were determined in the hippocampus of mice at 24 h after sham or CLP operation (*p<0.05, n=3). FIG. 1F depicts that neuron dendrite from sham and CLP mice were stained with Golgi-stain (left). Box plots of dendritic spine number in sham and CLP group were analyzed at 8 weeks post-surgery (right) (Scale bar=20 μm *P<0.05, n=3-4 mice/group).

FIG. 2A through FIG. 2G depict exemplary experimental data demonstrating that Neat1 expression is increased during sepsis. FIG. 2A and FIG. 2B depict exemplary experimental data demonstrating that Neat1 levels in brain tissues (FIG. 2A) and neurons (FIG. 2B) at different interval after CLP were assessed (*P<0.05, **P<0.01, n=3-6 mice/time point/group). FIG. 2C depicts data demonstrating that Neat1 levels in the hippocampus of sham and CLP mice were determined with RNA-FISH to visualize Neat1, neuron and nuclei at 24 hours after CLP (Scale bar=25 μm). FIG. 2D depicts data demonstrating that Neat1 positive neuronal cells in hippocampus were quantitated (*P<0.05, n=4). FIG. 2E depicts data demonstrating that N2a cells were incubated in normoxic or hypoxic conditions (1% O2) for 16 hours, Neat1 mRNA levels were detected by qRT-PCR (**P<0.01, n=4). FIG. 2F and FIG. 2G depict exemplary experimental data demonstrating that RNA-FISH assays (FIG. 2F) and quantitative data (FIG. 2G) for Neat1 positive N2a cells incubated in normoxic or hypoxic conditions (1% O2) for 16 hours. Arrows indicate Neat1 (Scale bar=8 μm, **P<0.01, n=4).

FIG. 3A through FIG. 3H depict exemplary experimental data demonstrating that Neat1 directly interacts with Hbb and regulates its expression. FIG. 3A depicts data demonstrating that Hbb was identified as an interacting target of Neat1 by MS analysis. FIG. 3B depicts data demonstrating that RNA pull-down assay followed by western blotting directly revealed interaction between Neat1 and Hbb in N2a cells (n=5-6). FIG. 3C and FIG. 3D depict exemplary experimental data demonstrating that RNA immunoprecipitation (RIP) assays were performed in N2a cells. Protein-RNA complexes immunoprecipitated by anti-Hbb or IgG were determined by qRT-PCR using primer for Neat1 (FIG. 3C) and the qRT-PCR products were analyzed by electrophoresis (FIG. 3D) (M: marker, *P<0.05 compared with the IgG group). FIG. 3E depicts data demonstrating that RNA-FISH/IF was performed to determine the colocalization of Neat1 and Hbb in N2a cells incubated in hypoxic (1% O₂) conditions for 6 hours. Arrows indicate the co-localized Neat1 and Hbb. nuclei (n=6, Scale bar=5 μm). FIG. 3F and FIG. 3G depict exemplary experimental data demonstrating that western blot (FIG. 3F) and immunofluorescence (FIG. 3G) Analysis of Hbb expression level in N2a cells incubated in normoxic or hypoxic (1% O₂) condition for 16 h (*P<0.05, n=3, nuclei, Hbb. Scale bar=33 μm). FIG. 3H depicts data demonstrating that the Hbb protein levels were determined in hippocampus of mice at 24 hours after sham and CLP operation (*P<0.05, n=3 mice/group).

FIG. 4A through FIG. 4I depict exemplary experimental data demonstrating that Neat1 stabilizes Hbb via inhibiting Hbb ubiquitination. FIG. 4A depicts data demonstrating that the Neat1 levels were measured in N2a cells transfected with Neat1 GapmeR. FIG. 4B and FIG. 4C depict exemplary experimental data demonstrating that the Hbb mRNA (FIG. 4B) and protein levels (FIG. 4C) in N2a cells after transfection with Neat1 GapmeR (*P<0.05, **P<0.01, n=4-5). FIG. 4D depicts data demonstrating that the Neat1 levels were determined in primary neuronal cells transfected with Neat1 GapmeR for 24 hours (*P<0.05, n=5-6). FIG. 4E and FIG. 4F depict exemplary experimental data demonstrating that the Hbb mRNA (FIG. 4E) and protein levels (FIG. 4F) in primary neuronal cells after transfection with Neat1 GapmeR for 24 hours (*P<0.05, n=3-4). FIG. 4G depicts data demonstrating that the N2a cells transfected with Neat1 GapmeR were treated with cycloheximide (CHX, 200 g/mL) for the indicated times and Hbb protein levels were determined. FIG. 4H depicts data demonstrating that the protein levels of Hbb in N2a cells transfected with control or Neat1 GapmeR and treated with MG-132 (5 μM) were determined by western blot assay. FIG. 4I depicts data demonstrating that the N2a cells transfected with control or Neat1 GapmeR were treated with MG-132 (5 μM) for 16 hours. Cell lysates were immunoprecipitated with antibodies against Hbb or IgG. The levels of ubiquitination were analyzed by western blot. Lower panel, input from cell lysates. IB, immunoblot.

FIG. 5A through FIG. 5G depict exemplary experimental data demonstrating that Neat1/Hbb axis suppresses PSD-95 expression and dendritic spine density. FIG. 5A depicts data demonstrating that the Neat1 levels were measured in N2a cells after transfection with Neat1 GapmeR for 48 hours (*P<0.05, n=4). FIG. 5B depicts data demonstrating that the PSD-95 protein levels were determined after transfection of N2a cells with Neat1 GapmeR for 48 hours (*P<0.05, n=6). FIG. 5C depicts data demonstrating that the protein levels of PSD-95 were detected after transfection of the primary neuronal cells with Neat1 GapmeR for 24 h (**P<0.01, n=3-4). FIG. 5D depicts data demonstrating that primary neurons were transfected with control or Neat1 GapmeR for 24 hours. The dendritic spine numbers were analyzed by immunostaining to label PSD-95 puncta and axons (*P<0.05, n=6, PSD-95, βIII-Tubulin, Scale bar=5 μm). FIG. 5E and FIG. 5F depict exemplary experimental data demonstrating that the protein levels of Hbb and PSD-95 were determined after transfection of the N2a cells (FIG. 5E) and primary neuron cells (FIG. 5F) with control or Hbb GapmeR (*P<0.05, **P<0.01, n=5-6).). FIG. 5G depicts data demonstrating that control or Hbb GapmeR were transfected into primary neuronal cells for 24 hours. The dendritic spine numbers were analyzed by immunostaining to label PSD-95 puncta and axons. (**P<0.01, n=6, PSD-95, βIII-Tubulin, Scale bar=5 μm).

FIG. 6A through FIG. 6F depict exemplary experimental data demonstrating that Neat1 deficiency attenuates anxiety-like behavior and cognitive dysfunction post sepsis. FIG. 6A depicts data demonstrating that the qRT-PCR was performed to measure Neat1 expression in hippocampus from wild-type (WT) and Neat1−/− mice (**P<0.01, n=3-4). FIG. 6B and FIG. 6C depict exemplary experimental data demonstrating that the WT (n=13) and Neat1−/− (n=12) mice were subjected to open field test at 2 weeks after CLP. FIG. 6B depicts representative tracks and heat map. FIG. 6C depicts data demonstrating the frequency, time in the center zone and the first time for the mice go to the center zone were analyzed (*P<0.05). FIG. 6D depicts WT (n=13) and Neat1−/− (n=11) mice were subjected to contextual fear conditioning test at 6 weeks after CLP and freezing behavior as a percent of freezing time was analyzed (*P<0.05). FIG. 6E depicts data demonstrating the protein level of Hbb and PSD-95 were analyzed in the hippocampus of WT and Neat1−/− mice at 24 hours after CLP (*P<0.05, n=3 mice/group). FIG. 6F depicts data demonstrating the neuron dendrite from WT and Neat1−/− mice at 8 weeks after CLP were stained with Golgi-stain and dendritic spine number were analyzed (*P<0.05, n=3 mice/group).

FIG. 7A through FIG. 7F depict exemplary experimental data demonstrating that Neat1 GapmeR ameliorated anxiety and cognitive impairment post sepsis. FIG. 7A depicts data demonstrating that mice were subjected to cecal ligation and puncture (CLP)—induced sepsis and treated with control and Neat1-GapmeR, the Neat1 levels and mRNA levels of c-fos and Bdnf in brain tissue were determined at 24 hours after CLP (*P<0.05, **P<0.01, n=4). FIG. 7B and FIG. 7C depict exemplary experimental data demonstrating that septic mice were treated with control (n=12) or Neat1 (n=14) GapmeR and subjected to open field test at 2 weeks after CLP. FIG. 7B depicts data demonstrating representative tracks and heat map are shown. FIG. 7C depicts data demonstrating the frequency and time for mice to enter the center zones as well as the first time for the mice to enter the center zone were analyzed (*P<0.05). FIG. 7D depicts data demonstrating that septic mice were treated with control (n=18) and Neat1 (n=15) GapmeR and subjected to contextual fear conditioning test at 6 weeks after CLP. Freezing behavior as a percent of freezing time was analyzed (*P<0.05). FIG. 7E depicts data demonstrating PSD-95 protein levels were analyzed in brain tissues of control and Neat1 GapmeR treated septic mice at 24 h after CLP (*P<0.05, n=3 mice/group). FIG. 7F depicts data demonstrating that neuron dendrite from sham mice, and septic mice treated with control or Neat1 GapmeR were stained with Golgi-stain and dendritic spine number were analyzed at 8 weeks post-surgery (*P<0.05, n=3 mice/group).

FIG. 8 depicts data demonstrating a schematic model for Neat1/Hbb axis regulates post-sepsis cognitive impairment. During sepsis, neurons are exposed to hypoxic environments, which results in upregulation of Neat1 levels. Neat1 directly interacts with Hbb and stabilizes its protein levels via inhibiting Hbb ubiquitination. Furthermore, Hbb suppression of PSD-95 expression results in decreased dendritic spine density, which are critical for cognition and memory. In addition, suppression of Neat1 via GapmeR promotes degradation of Hbb, resulting in increases of PSD-95 levels, reduction of synaptic dysfunction and mitigation of cognitive impairment post sepsis.

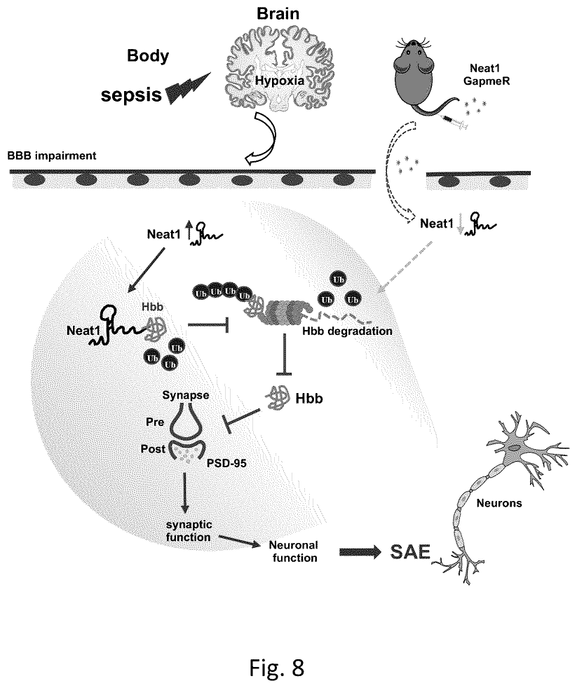

FIG. 9A through FIG. 9C depict exemplary experimental data demonstrating the survival rate of CLP-induced sepsis model and timeline of the experimental design. FIG. 9A depicts data demonstrating that cecal ligation and puncture (CLP)-induced sepsis resulted in 47% mortality over 7 days. FIG. 9B depicts a diagram of the timeline for the experiments in this study. Mice were subjected to sham or CLP surgery. The open field (OF) test was performed at 2 weeks after CLP, and contextual fear conditioning (CFC) test was performed at 6 weeks after CLP. Mice were sacrificed at 8 weeks after CLP and dendritic spine density were determined. FIG. 9C depicts a graphic depiction of CFC paradigm. Mice were subjected to a foot shock at 6 weeks after CLP and freezing behavior was monitored 24 hours after the foot shock.

FIG. 10A through FIG. 10C depict exemplary experimental data demonstrating the LncRNA expression levels in mouse brain tissues after CLP. The expression levels of Neat1 (FIG. 10A), HOTAIR (FIG. 10B) and Malat1 (FIG. 10C) in brain tissue were assessed 24 hours after sham or CLP (*P<0.05, **P<0.01, n=3-6 mice/group).

FIG. 11A through FIG. 11C depict exemplary experimental data demonstrating that inflammatory cytokines IL-1β, TNFα or LPS do not induce Neat1 expression in N2a cell. N2a cells were treated with IL-1β (40 ng/ml, FIG. 11A), TNF-α (20 ng/ml, FIG. 11B) or LPS (100 ng/ml, FIG. 11C) for 16 hours. Neat1 expression levels were determined by RT-PCR (n=3).

FIG. 12A through FIG. 12C depict exemplary experimental data demonstrating that hypoxia induced increases of Neat1 levels were mediated through HIF-2α dependent signaling pathway. FIG. 12A depicts data demonstrating that N2a cells were treated with siRNA against the HIF-2α and HIF-2α mRNA levels were analyzed by RT-PCR (*P<0.05, n=3). N2a cells were transfected with control or HIF-2a siRNA and expression levels of HIF-2α (FIG. 12B) and Neat1 (FIG. 12C) in the normoxia and hypoxia condition were determined by RT-PCR (*P<0.05, **P<0.01 compared with normoxia group, #P<0.05 compared with si-Ctrl hypoxia group, n=3).

FIG. 13A and FIG. 13B depict exemplary experimental data demonstrating that Hbb protein was not associated with Malat1. RNA immunoprecipitation (RIP) assays were performed in N2a cells. Protein-RNA complexes immunoprecipitated by anti-Hbb or control IgG were determined by qRT-PCR using primer for Malat1 (FIG. 13A) and the qRT-PCR products were analyzed by electrophoresis (FIG. 13B) (M: marker).

FIG. 14A through FIG. 14E depict exemplary experimental data demonstrating that Neat1 stabilizes Hbb via inhibiting Hbb ubiquitination. FIG. 14A depicts data demonstrating that the Neat1 levels were measured in N2a cells transfected with Neat1 GapmeR #2 (**P<0.01, n=3). FIG. 14B depicts data demonstrating that the Hbb protein levels in N2a cells after transfection with Neat1 GapmeR #2 (*P<0.05, n=3). FIG. 14C depicts data demonstrating that the Neat1 levels were determined in primary neuronal cells transfected with Neat1 GapmeR #2 for 24 hours (*P<0.05, n=6). FIG. 14D depicts data demonstrating that the Hbb protein levels in primary neuronal cells after transfection with Neat1 GapmeR #2 for 24 hours (*P<0.05, n=4-6). FIG. 14E depicts data demonstrating that the N2a cells transfected with control or Neat1 GapmeR #2 were treated with MG-132 (5 M) for 16 hours. Cell lysates were immunoprecipitated with antibodies against Hbb or IgG. The levels of ubiquitination were analyzed by western blot. Lower panel, input from cell lysates. IB, immunoblot.

FIG. 15A through FIG. 15C depict exemplary experimental data demonstrating that inhibition of Neat1 by GapmeR Neat1 #2 increases PSD-95 expression and dendritic spine density. FIG. 15A depicts data demonstrating that the PSD-95 protein levels were measured in N2a cells after transfection with Neat1 GapmeR #2 for 48 h (*P<0.05, n=5-6). FIG. 15B depicts data demonstrating that the protein levels of PSD-95 were detected after transfection of the primary neuronal cells with Neat1 GapmeR #2 for 24 hours (*P<0.05, n=3-4). FIG. 15C depicts data demonstrating that primary neurons were transfected with control or Neat1 GapmeR #2 for 24 hours. The dendritic spine numbers were analyzed by immunostaining to label PSD-95 puncta and axons (*P<0.05, n=6, PSD-95, βIII-Tubulin, Scale bar=5 μm).

FIG. 16 depicts data demonstrating the survival rate of CLP-induced sepsis model in wild-type and Neat1−/− mice. Survival curves of WT and Neat1−/− mice after cecal ligation and puncture (CLP) over 168 hours. Mortality rate for WT mice was 42% and for Neat1−/− mice was 55%.

FIG. 17A and FIG. 17B depict exemplary experimental data demonstrating the survival rate of CLP-induced septic mice treated with GapmeRs and the experimental design for behavior tests after Neat1 GapmeR treatment. FIG. 17A depicts data demonstrating that the cecal ligation and puncture (CLP) sepsis resulted in 57% mortality after treatment with control GapmeR over 7 days, and CLP mice treatment with Neat1 GapmeR resulted in 55% mortality. FIG. 17B depicts a graphic depiction of open field test and single-pairing CFC paradigm in the GapmeR treated septic mice.

FIG. 18 depicts data demonstrating the details of proteins that bound to Neat1 in lysed neuronal cells and their expression levels were altered 2-fold after CLP by LC-MS/MS analysis

FIG. 19A and FIG. 19B depict exemplary experimental data demonstrating the Neat1 levels are increased in AD patients compared to controls. Brain tissue from the hippocampus (FIG. 19A), and superior temporal gyrus (FIG. 19B) of patients with AD (N=28) and controls (N=15) were obtained from the Brain Bank at MUSC. Neat1 levels were determined by RT-PCR. *p<0.05 compared to control group.

FIG. 20A and FIG. 20B depict exemplary experimental data demonstrating the Neat1 levels are increased in 5×FAD mice. Mouse brain hippocampus (FIG. 20A) and cortex (FIG. 20B) were isolated from WT and littermate 5×FAD mice. Neat1 levels were determined by RT-PCR. N=5, p<0.05.

FIG. 21A and FIG. 21B depict exemplary experimental data demonstrating that the intrathecal injection of Neat1 Gapmer decrease Neat1 levels in the brain. 5 nmol/kg of Neat1 Gapmer were intrathecal injected into intrathecal space in WT mice. Neat1 levels in the hippocampus (FIG. 21A) and cortex (FIG. 21B) were determined at 7 days after injection. p<0.05, N=4-5 mice/group.

FIG. 22 depicts data demonstrating the effects of human Neat1 Gapmers on Neat1 levels in human brain pericytes. We have designed 5 human Neat1 Gapmers. Human brain pericytes were transfected with each Gapmer for 48 hours and Neat1 levels were determined by RT-PCR. **p<0.01, N=5.

### DETAILED DESCRIPTION

The invention is based, in part on the finding that Neat1 silencing via an antisense oligonucleotide GapmeR ameliorated anxiety-like behavior and cognitive impairment post sepsis. Thus, the invention is based on a previously unknown mechanism of the Neat1/Hbb axis in regulating neuronal dysfunction, and provides a novel treatment strategy for treatment of neurodegenerative diseases including, but not limited to, Alzheimer's Disease (AD), COVID-19 induced cognitive impairment, and Sepsis-associated encephalopathy (SAE).

Therefore in various embodiments, the invention provides compositions and methods for modulating the Neat1/Hbb axis through modulating Neat1 expression or activity. In some embodiments, the invention provides an antisense oligonucleotide GapmeR specific for Neat1 silencing.

### Definitions

Unless defined otherwise, all technical and scientific terms used herein have the same meaning as commonly understood by one of ordinary skill in the art to which this invention belongs.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of 20%, ±10%, ±5%, ±1%, or ±0.1% from the specified value, as such variations are appropriate to perform the disclosed methods.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of 20%, ±10%, ±5%, ±1%, or ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

The term “activate,” as used herein, means to induce or increase an activity or function, for example, about ten percent relative to a control value. Preferably, the activity is induced or increased by 50% compared to a control value, more preferably by 75%, and even more preferably by 95%. “Activate,” as used herein, also means to increase a molecule, a reaction, an interaction, a gene, an mRNA, and/or a protein's expression, stability, function or activity by a measurable amount or to increase entirely. Activators are compounds that, e.g., bind to, partially or totally induce stimulation, increase, promote, induce activation, activate, sensitize, or up regulate a protein, a gene, and an mRNA stability, expression, function and activity, e.g., agonists.

“Antisense” refers particularly to the nucleic acid sequence of the non-coding strand of a double stranded DNA molecule, or to a sequence which is substantially homologous to the non-coding strand. As defined herein, an antisense sequence is complementary to the sequence of a double stranded DNA molecule. It is not necessary that the antisense sequence be complementary solely to the coding portion of the coding strand of the DNA molecule. The antisense sequence may be complementary to regulatory sequences specified on the coding strand of a DNA molecule, which regulatory sequences control expression of the coding sequences.

The term “cancer” as used herein is defined as disease characterized by the abnormal growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer, sarcoma and the like.

As used herein, the term “cardiovascular disease” or “CVD,” generally refers to heart and blood vessel diseases, including atherosclerosis, coronary heart disease, cerebrovascular disease, and peripheral vascular disease. Cardiovascular disorders are acute manifestations of CVD and include myocardial infarction, stroke, angina pectoris, transient ischemic attacks, and congestive heart failure. Cardiovascular disease, including atherosclerosis, usually results from the build-up of cholesterol, inflammatory cells, extracellular matrix and plaque. As used herein, the term “coronary heart disease” or “CHD” refers to atherosclerosis in the arteries of the heart causing a heart attack or other clinical manifestation such as unstable angina.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

An “effective amount” or “therapeutically effective amount” of a compound is that amount of a compound which is sufficient to provide a beneficial effect to the subject to which the compound is administered. An “effective amount” of a delivery vehicle is that amount sufficient to effectively bind or deliver a compound.

As used herein, a “gapmer” is a region of a natural or nonnatural nucleotide sequence having one or more nucleosides that can bind to a target oligonucleotide. In some embodiments, the gapmer comprises a domain comprising one or a plurality of modified or unmodified deoxynucleotides. In some embodiments, the gapmer comprises a domain comprising one or a plurality of modified or unmodified ribonucleotides. In some embodiments, the gapmer hybridization to a target sequence induces cleavage of at least a portion of the target oligonucleotide by Rnase H. In some embodiments, the gapmer is an LNA gapmer comprising one or more LNA domain. In some embodiments, the gapmer is a chimeric antisense compound. In some embodiments, the gapmer comprises a DNA domain linked to an LNA domain. In some embodiments, the gapmer comprises a DNA domain flanked by two LNA domains. In some embodiments, the gapmer has an internal region having a plurality of nucleosides that support RNase H cleavage, positioned between external regions having one or more nucleosides. In some embodiments, the nucleosides comprising the internal region are chemically distinct from the nucleoside or nucleosides comprising the external regions. In certain embodiments, the target oligonucleotide comprises from about 5 to about 200, from about 5 to about 50, from about 10 to about 100, from about 10 to about 50, from about 10 to about 25, from about 15 to about 100, from about 15 to about 50, from about 5 to about 25, or from about 15 to about 25 nucleotides.

The term “inhibit,” as used herein, means to suppress or block an activity or function, for example, about ten percent relative to a control value. Preferably, the activity is suppressed or blocked by 50% compared to a control value, more preferably by 75%, and even more preferably by 95%. “Inhibit,” as used herein, also means to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down regulate a protein, a gene, and an mRNA stability, expression, function and activity, e.g., antagonists.

As used herein, an “instructional material” includes a publication, a recording, a diagram, or any other medium of expression which can be used to communicate the usefulness of a compound, composition, vector, or delivery system of the invention in the kit for effecting alleviation of the various diseases or disorders recited herein. Optionally, or alternately, the instructional material can describe one or more methods of alleviating the diseases or disorders in a cell or a tissue of a mammal. The instructional material of the kit of the invention can, for example, be affixed to a container which contains the identified compound, composition, vector, or delivery system of the invention or be shipped together with a container which contains the identified compound, composition, vector, or delivery system. Alternatively, the instructional material can be shipped separately from the container with the intention that the instructional material and the compound be used cooperatively by the recipient.

By the term “modulating,” as used herein, is meant mediating a detectable increase or decrease in the level of a response in a subject compared with the level of a response in the subject in the absence of a treatment or compound, and/or compared with the level of a response in an otherwise identical but untreated subject. The term encompasses perturbing and/or affecting a native signal or response thereby mediating a beneficial therapeutic response in a subject, preferably, a human.

As used herein, a “modulator of one or more Neat1 variants” is a compound that modifies the expression, activity or biological function of the Neat1 variant or RNA as compared to the expression, activity or biological function of the Neat1 variant or RNA in the absence of the modulator.

The terms “patient,” “subject,” “individual,” and the like are used interchangeably herein, and refer to any animal, or cells thereof whether in vitro or in vivo, amenable to the methods described herein. In certain non-limiting embodiments, the patient, subject or individual is a human.

A “therapeutic” treatment is a treatment administered to a subject who exhibits signs or symptoms of a disease or disorder, for the purpose of diminishing or eliminating those signs or symptoms.

As used herein, “treating a disease or disorder” means reducing the severity and/or frequency with which a sign or symptom of the disease or disorder is experienced by a patient.

The phrase “biological sample” as used herein, is intended to include any sample comprising a cell, a tissue, or a bodily fluid in which expression of a nucleic acid or polypeptide is present or can be detected. Samples that are liquid in nature are referred to herein as “bodily fluids.” Biological samples may be obtained from a patient by a variety of techniques including, for example, by scraping or swabbing an area of the subject or by using a needle to obtain bodily fluids. Methods for collecting various body samples are well known in the art.

As used herein, an “immunoassay” refers to any binding assay that uses an antibody capable of binding specifically to a target molecule to detect and quantify the target molecule.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific.

In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

A “coding region” of a gene consists of the nucleotide residues of the coding strand of the gene and the nucleotides of the non-coding strand of the gene.

“Complementary” as used herein to refer to a nucleic acid, refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

“Isolated” means altered or removed from the natural state. For example, a nucleic acid or a peptide naturally present in its normal context in a living animal is not “isolated,” but the same nucleic acid or peptide partially or completely separated from the coexisting materials of its natural context is “isolated.” An isolated nucleic acid or protein can exist in substantially purified form, or can exist in a non-native environment such as, for example, a host cell.

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA which is part of a hybrid gene encoding additional polypeptide sequence.

An “LNA gapmer,” as used herein refers to an oligonucleotide composed of LNA segments flanking a central DNA gap that can be phosphorothionated. In some embodiments, the central DNA gap is about 6 or more nucleotides, for example, from about 7 to about 10 nucleotides. In some embodiments, the central DNA gap is 11 or more nucleotides in length. In some embodiments, the LNA gapmer is from about 8 to about 120 nucleotides. In some embodiments, the LNA gapmer is from about 10 to about 100 nucleotides. In some embodiments, the LNA gapmer is from about 10 to about 80 nucleotides. In some embodiments, the LNA gapmer is from about 10 to about 60 nucleotides. In some embodiments, the LNA gapmer is from about 10 to about 40 nucleotides. In some embodiments, the LNA gapmer is from about 10 to about 30 nucleotides. In some embodiments, the LNA gapmer is from about 10 to about 25 nucleotides. In some embodiments, the LNA gapmer is from about 10 to about 20 nucleotides. In some embodiments, the LNA gapmer is from about 8 to about 30 nucleotides. In some embodiments, the LNA gapmer is from about 8 to about 20 nucleotides. In some embodiments, the LNA gapmer is from about 14 to about 16 nucleotides.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, “conjugated” refers to covalent attachment of one molecule to a second molecule.

The term “target domain” refers to a amino acid sequence or nucleic acid element or domain within a nucleic acid sequence (or polynucleotide sequence) that binds to an LNA gapmer either covalently or non-covalently when the LNA gapmer is in contact with the target domain in a biophysically effective amount. In some embodiments, the target domain consists of no more than about 5, 10, 15, 20, 25, 30, 35, 40, 45, 50, 60, 70, 80, 90, 100, 110, 120, 130, 140, 150, 160, 170, 180, 190, 200, 210, 220, 230, 240, 250 or more nucleotides in length. In some embodiments, the target domain is expressed by a cell, such as a human cell.

“Variant” as the term is used herein, is a nucleic acid sequence or an amino acid sequence that differs in sequence from a reference nucleic acid sequence or amino acid sequence respectively, but retains essential biological properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis. In some embodiments, the variant sequence is at least about 85%, 86%, 87%, 88%, 89%, 90%, 91%, 92%, 93%, 94%, 95%, 96%, 97%, 98%, or 99%, identical to the reference sequence.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

The present invention relates generally to compositions and methods for modulating the activity of the Neat1/Hbb axis for the treatment or prevention of neurodegenerative diseases or disorders. In one embodiment, the present invention provides compositions and methods for decreasing or inhibiting the expression or activity of Neat1.

In various embodiments, the present invention is directed to methods and compositions for treatment, inhibition, prevention, or reduction of neurodegenerative diseases and disorders.

In various embodiments, the compositions of the invention comprises a modulator of the level or activity of Neat1, or the level or activity of a regulator of Neat1.

### Compositions

In various embodiments, the present invention includes compositions for modulating the level or activity of Neat1 in a subject, a cell, a tissue, or an organ in need thereof.

Modulation of a gene, or gene product, can be assessed using a wide variety of methods, including those disclosed herein, as well as methods known in the art or to be developed in the future. That is, the routineer would appreciate, based upon the disclosure provided herein, that modulating the level or activity of a gene, or gene product, can be readily assessed using methods that assess the level of a nucleic acid encoding a gene product (e.g., mRNA, lncRNA), the level of gene product present in a biological sample, the activity of gene product present in a biological sample, or combinations thereof.

The modulator compositions and methods of the invention that modulate the level or activity of a gene, or gene product, include, but should not be construed as being limited to, a chemical compound, a protein, a peptide, a peptidomemetic, an antibody, a ribozyme, a small molecule chemical compound, a nucleic acid, a vector, an antisense nucleic acid molecule (e.g., siRNA, miRNA, GapmeR etc.), or combinations thereof. One of skill in the art would readily appreciate, based on the disclosure provided herein, that a modulator composition encompasses a chemical compound that modulates the level or activity of a gene, or gene product. Additionally, a modulator composition encompasses a chemically modified compound, and derivatives, as is well known to one of skill in the chemical arts.

Further, one of skill in the art would, when equipped with this disclosure and the methods exemplified herein, appreciate that modulators include such modulators as discovered in the future, as can be identified by well-known criteria in the art of pharmacology, such as the physiological results of modulation of the genes, and gene products, as described in detail herein and/or as known in the art. Therefore, the present invention is not limited in any way to any particular modulator composition as exemplified or disclosed herein; rather, the invention encompasses those modulator compositions that would be understood by the routineer to be useful as are known in the art and as are discovered in the future.

Further methods of identifying and producing modulator compositions are well known to those of ordinary skill in the art. Alternatively, a modulator can be synthesized chemically. Further, the person of skill in the art would appreciate, based upon the teachings provided herein, that a modulator composition can be obtained from a recombinant organism. Compositions and methods for chemically synthesizing modulators and for obtaining them from natural sources are well known in the art and are described in the art.

One of skill in the art will appreciate that a modulator can be administered as a small molecule chemical, a polypeptide, a peptide, an antibody, a nucleic acid construct encoding a protein, an antisense nucleic acid (e.g., siRNA, miRNA, GapmeR etc.), a nucleic acid construct encoding an antisense nucleic acid, or combinations thereof. Numerous vectors and other compositions and methods are well known for administering a small molecule chemical, a polypeptide, a peptide, an antibody, a nucleic acid construct encoding a protein, an antisense nucleic acid (e.g., siRNA, miRNA, GapmeR etc.), or a nucleic acid construct encoding an antisense nucleic acid to cells or tissues.

In one embodiment, the invention provides a generic concept for inhibiting Neat1. In one embodiment, the composition of the invention comprises an inhibitor of at least one of a Neat1 variant, Hbb or the interaction of Neat 1 lncRNA with Hbb. In one embodiment, the inhibitor is selected from the group consisting of a small interfering RNA (siRNA), shRNA, a microRNA, a guide RNA, a micro RNA, a GapmeR, an antisense nucleic acid, a ribozyme, an expression vector encoding a transdominant negative mutant, an intracellular antibody, a peptide and a small molecule.

### Nucleic Acids

In one embodiment, the composition of the invention comprises one or more antisense nucleic acid molecules. For example, in one embodiment, the one or more antisense nucleic acid molecules are specific for targeting Neat1 lncRNA or Hbb. Antisense oligonucleotides are DNA or RNA molecules that are complementary to some portion of a mRNA or lncRNA molecule. When present in a cell, antisense oligonucleotides hybridize to an existing mRNA or lncRNA molecule and inhibit translation into a gene product or promote degradation of the RNA molecule. Inhibiting the expression of a gene using an antisense oligonucleotide is well known in the art (Marcus-Sekura, 1988, Anal. Biochem. 172:289), as are methods of expressing an antisense oligonucleotide in a cell (Inoue, U.S. Pat. No. 5,190,931). The methods of the invention include the use of antisense oligonucleotide to diminish the amount of Neat1 activity or Neat1 lncRNA or Hbb. Contemplated in the present invention are antisense oligonucleotides that are synthesized and provided to the cell by way of methods well known to those of ordinary skill in the art. As an example, an antisense oligonucleotide can be synthesized to be between about 10 and about 100, more preferably between about 15 and about 50 nucleotides long. The synthesis of nucleic acid molecules is well known in the art, as is the synthesis of modified antisense oligonucleotides to improve biological activity in comparison to unmodified antisense oligonucleotides (Tullis, 1991, U.S. Pat. No. 5,023,243).

Similarly, the expression of a gene may be inhibited by the hybridization of an antisense molecule to a promoter or other regulatory element of a gene, thereby affecting the transcription of the gene. Methods for the identification of a promoter or other regulatory element that interacts with a gene of interest are well known in the art, and include such methods as the yeast two hybrid system (Bartel and Fields, eds., In: The Yeast Two Hybrid System, Oxford University Press, Cary, N.C.).

Alternatively, inhibition of Neat1 or Hbb can be accomplished through the use of an siRNA, shRNA, antisense oligonucleotide or ribozyme. Given the nucleotide sequence of the molecule, one of ordinary skill in the art could synthesize an antisense oligonucleotide or ribozyme without undue experimentation, provided with the disclosure and references incorporated herein.

In one embodiment, siRNA is used to decrease the level of at least one Neat1 variant or Hbb. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., 1998, Nature 391(19):306-311; Timmons et al., 1998, Nature 395:854; Montgomery et al., 1998, TIG 14 (7):255-258; David R. Engelke, Ed., RNA Interference (RNAi) Nuts & Bolts of RNAi Technology, DNA Press, Eagleville, PA (2003); and Gregory J. Hannon, Ed., RNAi A Guide to Gene Silencing, Cold Spring Harbor Laboratory Press, Cold Spring Harbor, NY (2003). Soutschek et al. (2004, Nature 432:173-178) describe a chemical modification to siRNAs that aids in systemic delivery. Optimizing siRNAs involves consideration of overall G/C content, C/T content at the termini, Tm and the nucleotide content of the 3′ overhang. See, for instance, Schwartz et al., 2003, Cell, 115:199-208 and Khvorova et al., 2003, Cell 115:209-216. Therefore, the present invention also includes methods of decreasing levels of at least one Neat1 variant at the protein level using RNAi technology.

In certain embodiments, the modulators described herein comprise short hairpin RNA (shRNA) molecules. shRNA molecules are well known in the art and are directed against the mRNA of a target, thereby decreasing the expression of the target. In certain embodiments, the encoded shRNA is expressed by a cell, and is then processed into siRNA. For example, in certain instances, the cell possesses native enzymes (e.g., dicer) that cleaves the shRNA to form siRNA.

In other related aspects, the invention includes an isolated nucleic acid encoding an inhibitor, wherein an inhibitor such as an siRNA, shRNA, GapmeR or antisense molecule, inhibits at least one Neat1 variant, a derivative thereof, a regulator thereof, or a downstream effector thereof.

In some embodiments, the inhibitor of the disclosure comprises an oligonucleotide molecule comprising a gapmer domain comprising a sequence sufficiently complementary to a mammalian NEAT1 mRNA expressed by the cell such that the DNA gap domain hybridizes to the mRNA target sequence of the eukaryotic cell and degrades the mRNA, thereby reducing expression of the one or plurality of NEAT1 mRNA target sequences. In one embodiment, the DNA gap domain comprises at least one modified nucleotide.

In some embodiments, the inhibitor of the disclosure comprises an oligonucleotide molecule comprising a gapmer domain comprising a sequence sufficiently complementary to a mammalian NEAT1 lncRNA expressed by the cell such that the DNA gap domain hybridizes to the lncRNA target sequence of the eukaryotic cell and degrades the lncRNA, thereby reducing expression of the one or plurality of NEAT1 mRNA target sequences. In one embodiment, the DNA gap domain comprises at least one modified nucleotide.

In certain embodiments, the modification of the nucleotide in the DNA gap domain is one or more of 2′-O-methyl, 2′-O-fluoro, or phosphorothioate. In certain embodiments, the nucleotide is modified at the 2′ position of the sugar moiety. In certain embodiments, the modification at the 2′ position of the sugar moiety is 2′-O-methyl or 2′-O-fluoro. In certain embodiments, the nucleotide is modified at the 3′ position of the sugar moiety. In certain embodiments, the modification at the 3′ position of the sugar moiety is phosphorothioate. In certain embodiments, the nucleotide is modified at both the 2′ position of the sugar moiety and at the 3′ position of the sugar moiety. In certain embodiments, the nucleotide is not modified at the 2′ position of the sugar moiety. In certain embodiments, the nucleotide is not modified at the 3′ position of the sugar moiety.

In one embodiment the GapmeR molecule comprises at least one nucleotide modification. In some embodiments, the nucleotide modification is at least one locked nucleic acid (LNA) connecting adjacent nucleotides. Other modifications include but are not limited to, 2′-modified RNA phosphoramidites (e.g., 2′-OMe), 2′-methoxy (2′-O—CH3), 2′-aminopropoxy (2′-OCH2CH2CH2NH2), 2′-O-methoxyethyl (2MOE), and 2′-fluoro (2′-F). Modifications may be made at any position on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar.

Oligonucleotides may also include a modified thioester group on the 2′, 3′ and/or 5′ nucleoside. Such modifications in the 5′ carbon of the ribose sugar also for formation of single 5′-S-thioester linkages between nucleotides in a synthetic nucleotide sequence. In any 3′ or 5′ linkage between nucleotides any one or both positions may create a series of linkages between nucleotides. The linkages at the 2′ or 3′ can create thioester bond, phosphorothioriate linkages between two or a plurality of nucleosides in the oligonucleotide.

Strategically placed sulfur atoms in the backbone of nucleic acids have found widespread utility in probing of specific interactions of proteins, enzymes and metals. In some embodiments, sulfur replacement for oxygen may be carried out at the 2′-position of RNA and in the 3′-5′-positions of RNA and of DNA. In some embodiments, linkers of any cyclic or acyclic hydrocarbon chains of varying length may be incorporated into the nucleic acid. In some embodiments, linkers of the disclosure comprise one or a plurality of: branched or non-branched alkyl, hydroakyl, hydroxyl, halogen, metal, nitrogen, or other atoms.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Certain of these nucleobases are particularly useful for increasing the binding affinity of the oligomeric compounds of the disclosure. These include 5-substituted pyrimidines, 6-azapyrimidines and N-2, N-6 and 0-6 substituted purines, including 2-aminopropyladenine, 5-propynyluracil and 5-propynylcytosine.

In one embodiment, the GapmeR oligonucleotide comprises between about 10 to about 250 nucleotides. In one embodiment, the oligonucleotide comprises between about 20 to about 100 nucleotides. In one embodiment, the nucleic acid disclosed herein comprises from about 6 to about 120 nucleotides. In one embodiment, the nucleic acid disclosed herein comprises from about 10 to about 20 nucleotides. In some embodiments, the nucleic acid sequence comprises at least two domains, an LNA domain and a DNA gap domain. In some embodiments, the nucleic acid sequence comprises at least three domains, two LNA domains and a DNA gap domain.

In some embodiments, the oligonucleotides have one or more locked nucleic acids (LNA), which increase the binding affinity of GapmeR to the target RNA. In some embodiments, the GapmeR comprises at least 2 LNA on each of the 3′ and 5′ end of the GapmeR sequence flanking a phosphorothioated DNA region. In some embodiments, the GapmeR comprises at least 3 LNA on each of the 3′ and 5′ end of the GapmeR sequence flanking a phosphorothioated DNA region.

In some embodiments, the oligonucleotides have one or more phosphorothioated LNA. In some embodiments, the GapmeR comprises at least 2 phosphorothioated LNA on each of the 3′ and 5′ end of the GapmeR sequence flanking a phosphorothioated DNA region. In some embodiments, the GapmeR comprises at least 3 phosphorothioated LNA on each of the 3′ and 5′ end of the GapmeR sequence flanking a phosphorothioated DNA region.

In one embodiment, the antisense molecule is a GapmeR specific for Neat1 lncRNA. In some embodiments, the GapmeR has a sequence of: PGP-21 DNA

Human Gapmer-1
(SEQ ID NO: 1)
5′-+C*+A*+A*G*G*A*A*A*G*T*C*A*T*+C*+G*+C-3′
Human Gapmer-2
(SEQ ID NO: 2)
5′-+A*+A*+T*A*G*A*C*G*T*G*A*G*T*+G*+G*+A-3′
Human Gapmer-3
(SEQ ID NO: 3)
5′-+A*+T*+C*G*A*C*C*A*A*A*C*A*C*+A*+G*+A-3′
Human Gapmer-4
(SEQ ID NO: 4)
5′-+C*+A*+G*G*C*C*G*A*G*C*G*A*A*+A*+A*+T-3′
Human Gapmer-5
(SEQ ID NO: 5)
5′-+A*+C*+G*T*G*A*A*T*A*G*A*C*A*+G*+A*+T-3′
wherein each “*” = a Phosphorothioated DNA base:
G*, A*, T*, C*, and wherein each “+” = a LNA modified nucleotide.

In one embodiment, the inhibitor of the invention is an antisense molecule. Antisense molecules of the invention may be made synthetically and then provided to the cell. Antisense oligomers of between about 10 to about 30, and more preferably about 15 nucleotides, are preferred, since they are easily synthesized and introduced into a target cell. Synthetic antisense molecules contemplated by the invention include oligonucleotide derivatives known in the art which have improved biological activity compared to unmodified oligonucleotides (see U.S. Pat. No. 5,023,243).

Ribozymes and their use for inhibiting gene expression are also well known in the art (see, e.g., Cech et al., 1992, J. Biol. Chem. 267:17479-17482; Hampel et al., 1989, Biochemistry 28:4929-4933; Eckstein et al., International Publication No. WO 92/07065; Altman et al., U.S. Pat. No. 5,168,053). Ribozymes are RNA molecules possessing the ability to specifically cleave other single-stranded RNA in a manner analogous to DNA restriction endonucleases. Through the modification of nucleotide sequences encoding these RNAs, molecules can be engineered to recognize specific nucleotide sequences in an RNA molecule and cleave it (Cech, 1988, J. Amer. Med. Assn. 260:3030). A major advantage of this approach is the fact that ribozymes are sequence-specific.

There are two basic types of ribozymes, namely, tetrahymena-type (Hasselhoff, 1988, Nature 334:585) and hammerhead-type. Tetrahymena-type ribozymes recognize sequences which are four bases in length, while hammerhead-type ribozymes recognize base sequences 11-18 bases in length. The longer the sequence, the greater the likelihood that the sequence will occur exclusively in the target mRNA species. Consequently, hammerhead-type ribozymes are preferable to tetrahymena-type ribozymes for inactivating specific mRNA species, and 18-base recognition sequences are preferable to shorter recognition sequences which may occur randomly within various unrelated mRNA molecules.

In one embodiment of the invention, a ribozyme is used to inhibit at least one Neat1 variant. Ribozymes useful for inhibiting the expression of a target molecule may be designed by incorporating target sequences into the basic ribozyme structure which are complementary, for example, to the mRNA sequence of at least one Neat1 variant of the present invention. Ribozymes targeting at least one Neat1 variant may be synthesized using commercially available reagents or they may be genetically expressed from DNA encoding them.

In one embodiment, the inhibitor of at least one Neat1 variant may comprise one or more components of a CRISPR-Cas system, where a guide RNA (gRNA) targeted to a gene encoding at least one Neat1 variant or Hbb, and a CRISPR-associated (Cas) peptide form a complex to induce mutations within the targeted gene. In one embodiment, the inhibitor comprises a gRNA or a nucleic acid molecule encoding a gRNA. In one embodiment, the inhibitor comprises a Cas peptide or a nucleic acid molecule encoding a Cas peptide.

When the inhibitor of the invention is a small molecule, a small molecule antagonist may be obtained using standard methods known to the skilled artisan. Such methods include chemical organic synthesis or biological means. Biological means include purification from a biological source, recombinant synthesis and in vitro translation systems, using methods well known in the art.

Combinatorial libraries of molecularly diverse chemical compounds potentially useful in treating a variety of diseases and conditions are well known in the art as are method of making the libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development.

In a general method for small library synthesis, an activated core molecule is condensed with a number of building blocks, resulting in a combinatorial library of covalently linked, core-building block ensembles. The shape and rigidity of the core determines the orientation of the building blocks in shape space. The libraries can be biased by changing the core, linkage, or building blocks to target a characterized biological structure (“focused libraries”) or synthesized with less structural bias using flexible cores.

In other related aspects, the invention includes an isolated peptide inhibitor that inhibits at least one Neat1 variant, Hbb, or a combination thereof. For example, in one embodiment, the peptide inhibitor of the invention inhibits at least one Neat1 variant, Hbb, or a combination thereof directly by binding to at least one Neat1 variant or Hbb thereby preventing the normal functional activity of at least one Neat1 variant or Hbb. In another embodiment, the peptide inhibitor of the invention inhibits at least one Neat1 variant by competing with at least one endogenous Neat1 variant (e.g., inhibiting the interaction of at least on Neat1 variant with Hbb). In yet another embodiment, the peptide inhibitor of the invention inhibits the activity of at least one Neat1 variant by acting as a transdominant negative mutant.

### Substrates

The present invention provides a scaffold or substrate composition comprising a modulator of the invention, an isolated nucleic acid of the invention, a cell expressing the modulator of the invention, or a combination thereof. For example, in one embodiment, a modulator of the invention, an isolated nucleic acid of the invention, a cell a cell expressing the modulator of the invention, or a combination thereof is incorporated within a scaffold. In another embodiment, a modulator of the invention, an isolated nucleic acid of the invention, a cell expressing the modulator of the invention, or a combination thereof is applied to the surface of a scaffold. The scaffold of the invention may be of any type known in the art. Non-limiting examples of such a scaffold includes a, hydrogel, electrospun scaffold, foam, mesh, sheet, patch, and sponge.

### Pharmaceutical Compositions

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions that are useful in the methods of the invention may be prepared, packaged, or sold in formulations suitable for intrathecal, ophthalmic, oral, rectal, vaginal, parenteral, topical, pulmonary, intranasal, buccal, intratumoral, or another route of administration. Other contemplated formulations include projected nanoparticles, liposomal preparations, resealed erythrocytes containing the active ingredient, and immunologically-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents, including, for example, chemotherapeutics, immunosuppressants, corticosteroids, analgesics, and the like.

Controlled- or sustained-release formulations of a pharmaceutical composition of the invention may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intrathecal, intraocular, intravitreal, subcutaneous, intraperitoneal, intramuscular, intrasternal injection, intratumoral, and kidney dialytic infusion techniques. In one embodiment, the method of administration is through intrathecal injection.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The disclosure also relates to pharmaceutical compositions comprising: (i) one or nucleic acid sequences disclosed herein or one or more pharmaceutically acceptable salts thereof; and (ii) a pharmaceutically acceptable carrier. The term “pharmaceutically acceptable salts” refers to physiologically and pharmaceutically acceptable salts of the nucleic acid sequences of the disclosure: i.e., salts that retain the desired biological activity of the nucleic acid sequences and do not impart undesired toxicological effects thereto.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, for example, Berge et al., “Pharmaceutical Salts,” J. of Pharnut Sci., 1977, 66:1). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present disclosure. As used herein, a “pharmaceutical addition salt” includes a pharmaceutically acceptable salt of an acid form of one of the components of the compositions of the disclosure. These include organic or inorganic acid salts of the amines. In some embodiments, a pharmaceutically acceptable salt is selected from one or a combination of hydrochlorides, acetates, salicylates, nitrates and phosphates.

Other suitable pharmaceutically acceptable salts are well known to those skilled in the art and include basic salts of a variety of inorganic and organic acids, such as, for example, with inorganic acids, such as for example hydrochloric acid, hydrobromic acid, sulfuric acid or phosphoric acid; with organic carboxylic, sulfonic, sulfo or phospho acids or N-substituted sulfamic acids; for example acetic acid, propionic acid, glycolic acid, succinic acid, malefic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2phenoxybenzoic acid, 2-acetoxybenzoic acid, embonic acid, nicotinic acid or isonicotinic acid; and with amino acids, such as the 20 alpha-amino acids involved in the synthesis of proteins in nature, for example glutamic acid or aspartic acid, and also with phenylacetic acid, methanesulfonic acid, ethanesulfonic acid, 2-hydroxyethanesulfonic acid, ethane-1,2-disulfonic acid, benzenesulfonic acid, 4-methylbenzenesulfonic acid, naphthalene-2-sulfonic acid, naphthalene-1,5-disulfonic acid, 2- or 3-phosphoglycerate, glucose-6phosphate, N-cyclohexylsulfamic acid (with the formation of cyclamates), or with other acid organic compounds, such as ascorbic acid. Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible.

For oligonucleotides, examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, malefic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 nanometers, and preferably from about 1 to about 6 nanometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 nanometers and at least 95% of the particles by number have a diameter less than 7 nanometers. More preferably, at least 95% of the particles by weight have a diameter greater than 1 nanometer and at least 90% of the particles by number have a diameter less than 6 nanometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In one embodiment of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations that are useful include those that comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in the inventive formulations. Excipients such as cocoa butter and suppository waxes, coloring agents, coating agents, sweetening, flavoring, and perfuming agents can be present in the composition, according to the judgment of the formulator.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include, but are not limited to, natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g. Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof.

Exemplary binding agents include, but are not limited to, starch (e.g. cornstarch and starch paste); gelatin; sugars (e.g. sucrose, glucose, dextrose, dextrin, molasses, lactose, lactitol, mannitol); natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum), and larch arabogalactan); alginates; polyethylene oxide; polyethylene glycol; inorganic calcium salts; silicic acid; polymethacrylates; waxes; water; alcohol; etc.; and combinations thereof.

Additionally, the molecules may be delivered using a sustained-release system, such as semipermeable matrices of solid polymers containing the therapeutic agent. Various forms of sustained-release materials have been established and are well known by those skilled in the art. Sustained-release capsules may, depending on their chemical nature, release the molecules for a few weeks up to over 100 days. Depending on the chemical nature and the biological stability of the chimeric molecules, additional strategies for molecule stabilization may be employed.

Nucleic acids may be included in any of the above-described formulations as the free acids or bases or as pharmaceutically acceptable salts. Pharmaceutically acceptable salts are those salts that substantially retain the biologic activity of the free bases and which are prepared by reaction with inorganic acids. Pharmaceutical salts tend to be more soluble in aqueous and other protic solvents than are the corresponding free base forms.

In addition to the formulations described previously, the molecules may also be formulated as a depot preparation. Thus, the molecules may be formulated with suitable polymeric or hydrophobic materials (for example as an emulsion in an acceptable oil) or ion exchange resins, or as sparingly soluble derivatives, for example, as a sparingly soluble salt.

In some embodiments, the composition or pharmaceutical composition comprises any nucleic acid disclosed herein or its salt and one or more additional therapies. In some embodiments, the pharmaceutical composition comprises any one or plurality of nucleic acids disclosed herein or its salt or variant thereof and/or one or more therapies is administered to the subject before, contemporaneously with, substantially contemporaneously with, or after administration of the pharmaceutical composition.

Compositions of the disclosure include pharmaceutical compositions comprising: a particle comprising any of the nucleic acid sequences disclosed herein, or pharmaceutically acceptable salts thereof; and a pharmaceutically acceptable carrier. In some embodiments, the pharmaceutically acceptable carrier is distilled water or saline. In preferred embodiments, the pharmaceutically acceptable carrier is free of RNase/DNase.

As used herein, a “particle” refers to any entity having a diameter of less than 100 microns (μm). Typically, particles have a longest dimension (e.g. diameter) of 1000 nm or less. In some embodiments, particles have a diameter of 300 nm or less. In some embodiments, nanoparticles have a diameter of 200 nm or less. In some embodiments, nanoparticles have a diameter of 100 nm or less. In general, particles are greater in size than the renal excretion limit, but are small enough to avoid accumulation in the liver. In some embodiments, a population of particles may be relatively uniform in terms of size, shape, and/or composition. In general, inventive particles are biodegradable and/or biocompatible. Inventive particles can be solid or hollow and can comprise one or more layers. In some embodiments, particles are spheres, spheroids, flat, plate-shaped, cubes, cuboids, ovals, ellipses, cylinders, cones, or pyramids. In some embodiments, particles can be a matrix of polymers. In some embodiments, the matrix is cross-linked. In some embodiments, formation of the matrix involves a cross-linking step. In some embodiments, the matrix is not substantially cross-linked. In some embodiments, formation of the matrix does not involve a cross-linking step. In some embodiments, particles can be a non-polymeric particle (e.g. a metal particle, quantum dot, ceramic, inorganic material, bone, etc.). Components of the pharmaceutical compositions disclosed herein may comprise particles or may be microparticles, nanoparticles, liposomes, and/or micelles comprising one or more disclosed nucleic acid sequences or conjugated to one or more disclosed nucleic acids. As used herein, the term “nanoparticle” refers to any particle having a diameter of less than 1000 nm. In some embodiments, the particle is an exosome.

The practice of the present disclosure employs, unless otherwise indicated, conventional techniques of immunology, biochemistry, chemistry, molecular biology, microbiology, cell biology, genomics and recombinant DNA, which are within the skill of the art.

### Therapeutic Methods

The present disclosure also relates to a method of modulating or inhibiting expression of Neat1 in a subject. In one embodiment, the invention provides a method of altering a human cell by transfecting the human cell with a GapmeR disclosed herein with a DNA gap sequence sufficiently complementary to Neat1 lncRNA such that the DNA gap domain hybridizes to the lncRNA target sequence and the lncRNA is degraded, thereby reducing expression of Neat1. In one embodiment, the GapmeR includes between about 10 to about 250 nucleotides. In one embodiment, the GapmeR includes between about 20 to about 100 nucleotides.

In some embodiments, the method comprises administering to the subject a therapeutically effective amount of one or a combination of any composition described herein, and/or any pharmaceutical composition described herein.

In one embodiment, the invention provides methods of decreasing Neat1 activity, or expression, or decreasing the level of Neat1 lncRNA such that the modulation produces a therapeutic effect in a subject, or group of subjects. A therapeutic effect is one that results in an amelioration in the symptoms, or progression of a disease or disorder. In one embodiment, the disease or disorder is a neurodegenerative disease or disorder.

Examples of neurodegenerative diseases or disorders which may be treated or prevented by the compositions and methods of this invention include, but are not limited to septic induced cognitive impairment, COVID-19 induced cognitive impairment, mild cognitive impairment (MCI), sepsis-associated encephalopathy (SAE), frontotemporal dementia (FTD), epilepsy, traumatic brain injury, schizophrenia, polyQ disorders such as SCA1, SCA2, SCA3, SCA6, SCA7, SCA17, Huntington's disease, Dentatorubral-pallidoluysian atrophy (DRPLA), Alzheimer's disease, Parkinson's disease, amyotrophic lateral sclerosis (ALS), transmissible spongiform encephalopathies (prion disease), tauopathies, and Frontotemporal lobar degeneration (FTLD).

In one embodiment, the method comprises administering a composition described herein to a subject having, or having symptoms indicative of, a neurodegenerative disease or disorder. In one embodiment, the method comprises administering a composition described herein to a subject having, or having symptoms indicative of, a neurodegenerative disease or disorder. In one embodiment, the method comprises administering a composition described herein to a subject having sepsis or an infection that can lead to sepsis. In one embodiment, the method comprises administering a composition described herein to a subject having septic-induced or sepsis-associated cognitive impairment. In certain embodiments, the composition is administered to the subject via intrathecal injection.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: lncRNA Neat1 Regulates Neuronal Dysfunction Post Sepsis Via Stabilization of Hemoglobin Subunit Beta

Despite their frequency and profound impact on the health and well-being of sepsis survivors, little is known about the mechanisms underlying post-septic cognitive and psychiatric sequelae. This knowledge gap has significantly impacted the development of effective preventative and therapeutic strategies to address this major health concern. An important role of the lncRNA Neat1 was identified in the modulation of neuronal synaptic density and neuropsychiatric dysfunction among murine survivors of experimental sepsis. Specifically, it was shown that CLP-induced sepsis replicated clinical cognitive impairments, including anxiety-like behavior and long-term cognitive deficits in the mouse model, and increased the Neat1 expression in brain tissue, especially in neuronal cells. Furthermore, Neat1 mediated PSD-95 expression by interacting with Hbb protein and regulated dendritic spine density, which is associated with cognition and learning. Deficiency of Neat1 was sufficient to protect post-sepsis cognitive function in Neat1^{-/-} mice. Further, it was found that inhibition of Neat1 using systemic Neat1 GapmeR ameliorated sepsis-related dendritic spine loss and reduced cognitive dysfunction.

Sepsis survivors develop psychiatric sequelae and long-term cognitive impairment following discharge from Intensive care units (ICU), including anxiety, depression, alterations in memory, attention, concentration and/or global cognitive decline, which has a profound impact on their quality of life (Hopkins et al., 2005, Am J Respir Crit Care Med 171, 340-347). The use of behavioral testing in animal models of sepsis is a potentially valuable strategy to understand the onset and the time-course of the mental health problems and cognitive dysfunction associated with sepsis. Open field tests were frequently used for anxiety-like behavior, and contextual fear conditioning tests along with other inhibitory avoidance tests were widely used for assessing hippocampus-dependent memory (Savi et al., 2021, Neurosci Biobehav Rev 124, 386-404). Sepsis-induced by LPS and CLP are the most frequently used models, and both were effective in inducing short- and long-term behavioral impairment (Savi et al., 2021, Neurosci Biobehav Rev 124, 386-404). Compared with the LPS model, the CLP model more closely resembles the progression and characteristics of human sepsis (Dejager et al., 2011, Trends Microbiol 19, 198-208), and it could be a useful tool to study cognitive impairment, anxiety and depression, after sepsis, as well as the mechanisms associated with the recovery from such disorders (Savi et al., 2021, Neurosci Biobehav Rev 124, 386-404). In agreement with previous studies (Denstaedt et al., 2020, Shock 54, 78-86), it was demonstrated that sepsis-surviving mice show persistent impairment since anxiety-like behavior persists for at least 2 weeks after CLP, and memory impairment can be observed at least 6 weeks after sepsis and dendritic spin density remain decreased at 8 weeks after sepsis. These data suggest that the CLP sepsis model may recapitulate human SAE and reinforce the necessity of developing SAE targeted therapy to improve the quality of life for sepsis survivors. In addition, synapses are particularly vulnerable in neurodegenerative diseases and neurological disorders, and LPS decreases synaptic proteins and contributes to the memory deficit (Moraes et al., 2015, Mol Neurobiol 52, 653-663), which is consistent with data obtained from the CLP model. PSD-95, a major component responsible for synaptic maturation that regulates dendritic spines and developing synapses in the hippocampus, has been recently associated with neuropsychiatric disorders and reduction of PSD-95 was observed in septic mice (Huang et al., 2020, Brain Behav Immun 84, 242-252). Our data uncovered a novel mechanism that the Neat1/Hbb axis regulated PSD-95 levels and dendritic spine density in SAE.

Neat1 is an essential component of nuclear paraspeckles (Anantharaman et al., 2016, Sci Rep 6, 34043), which consist of ribonucleoprotein complexes formed around Neat1 (Yamazaki et al., 2018, Mol Cell 70, 1038-1053 e1037). Previous studies investigating Neat1 in the context of epilepsy have reported that, in the excitotoxic conditions of this neurodegenerative disorder, activity-dependent down-regulation of NEAT1 expression is impaired (Barry et al., 2017, Sci Rep 7, 40127). However, observations from studies on other neurodegenerative diseases would suggest that NEAT1 up-regulation is deleterious to neuronal survival (Liu et al., 2018, Clin Exp Pharmacol Physiol 45, 841-848). Moreover, another study found that increased NEAT1 expression might play a notable role in the age-related decline of hippocampus-dependent memory formation (Butler et al., 2019, Sci Signal 12, (588):eaaw9277). Therefore, abnormal Neat1 expression and/or regulation might underlie at least some of the symptoms and phenotypes in common neurological diseases. Interestingly, ample evidence also indicates that Neat1 participates in sepsis-induced organ injury in mice (Chen et al., 2018, Int Immunopharmacol 59, 252-260; Wang et al., 2019, Eur Rev Med Pharmacol Sci 23, 4898-4907; Zhang et al., 2019, Int Immunopharmacol 75, 105731). However, until now, the role of Neat1 in sepsis-associated neuronal dysfunction has been unknown. Since hypoxia may occur in brain tissue during sepsis, a key regulator of the transcriptional responses to hypoxia is hypoxia-inducible factor (HIF) (Kaelin et al., 2008, Mol Cell 30, 393-402). Recently, HIF has been shown to play a critical role in regulating the non-coding transcriptional response (Shih et al., 2017, J Biomed Sci 24, 53). It is reported that hypoxia can induce Neat1 through HIF-2α-mediated transcriptional activation (Choudhry et al., 2015, Oncogene 34, 4546; Choudhry et al., 2016, Brief Funct Genomics 15, 174-185), and it was found that inhibition of HIF-2α attenuated hypoxia-induced increases of Neat1 in N2a cells. Historically, Neat1 was thought to be exclusive to the nucleus and mainly function as a transcriptional regulator. In this study, it was found that Neat1 was present both in the nucleus and the cytoplasm and exerted post-translational regulation of Hbb via direct binding and inhibition of ubiquitination. Given a report suggesting that Hbb may be a part of a mechanism linking neuronal energetics with epigenetic changes and may function by supporting neuronal metabolism (Brown et al., 2016, J Mol Neurosci 59, 1-17), Hbb was further analyzed. Silencing of Neat1 or Hbb both led to increased PSD-95 and dendritic spines as well as reductions of post-sepsis neuropsychiatric sequelae. The exact role of hemoglobin in neurons is debated although changes in its expression and subcellular localization have been associated with a few neurodegenerative disorders including multiple sclerosis, Alzheimer's disease and Parkinson's disease (Brown et al., 2016, J Mol Neurosci 59, 1-17; Singhal et al., 2018, Mol Neurobiol 55, 8051-8058; Ferrer et al., 2011, J Alzheimers Dis 23, 537-550). This study's observations combined with emerging data suggesting the importance of cell-free hemoglobin in sepsis-related organ failure, provide a strong rationale for ongoing investigation into the role of Hbb in SAE. Previous studies demonstrated that septic mice exhibited decreases of β2-adrenoceptor (β2-AR), increases of neuroinflammation, and decreases of BDNF and PSD-95 expression (Zong et al., 2019, Front Cell Neurosci 13, 293) which are consistent with the findings presented here. It was demonstrated that treatment with β2-AR agonist clenbuterol ameliorated sepsis-induced cognitive impairment, reduced proinflammatory cytokine and up-regulated BDNF, PSD-95 levels (Zong et al., 2019, Front Cell Neurosci 13, 293). Whether activation of β2-AR could alter Neat1 expression remains to be further investigated.

A comprehensive analysis of the molecular perturbations produced by Neat1 in neuronal cells provides the first evidence of its potential as a therapeutic target in neurons of SAE. To efficiently target Neat1, a novel LNA GapmeR antisense oligonucleotide was used which serves as a unique tool to efficiently knock down lncRNAs (Lennox et al., 2016, Nucleic Acids Res 44, 863-877). LNA GapmeRs have a central DNA gap that binds the RNA target, and triggers its RNase H-dependent degradation; the presence of phosphorothioate confers nuclease resistance in bodily fluids (Stein et al., 2010, Nucleic Acids Res 38, e3), while LNA increases affinity to the target (Roux et al., 2017, Methods Mol Biol 1468, 11-18). LNA GapmeRs are becoming an attractive therapeutic modality to target undruggable pathways in vivo. Prior studies suggest that, in physiological conditions, LNA GapmeRs cannot pass through the blood-brain barrier to reach the brain by systemic administration (Straarup et al., 2010, Nucleic Acids Res 38, 7100-7111). However, this investigation capitalized on the known blood-brain barrier interruption associated with sepsis which allowed intravenously injected Neat1 GapmeRs to enter the brain and impart beneficial effects on both synaptic density and post-sepsis neuropsychiatric symptoms. These findings support the therapeutic potential of LNA GapmeRs in human illness and serve as the basis of ongoing translational studies in SAE.

In summary, the interaction between Neat1 and Hbb was identified, which together regulate dendritic spine density and impact cognitive dysfunctions after sepsis. As a result, Neat1 and Hbb may become a potential target for diagnosis and a treatment strategy for sepsis-associated acute brain dysfunction. Furthermore, these roles may extend to other sources of acute brain dysfunction including COVID-19. Finally, the first evidence of mitigating SAE through a novel antisense oligonucleotide GapmeR based reduction of Neat1 is reported, which could lead to a novel therapeutic strategy to combat SAE.

The Materials and Methods are now described

### Animals

Male and female C57BL/6 Mice (8-12 weeks old) were purchased from the Jackson Laboratory and housed in a pathogen-free environment. Neat1 knockout mice were generated (Nakagawa et al., 2014, Development 141, 4618-4627) and provided. Male and female Neat 1 knockout mice (8-12 weeks) and littermate WT mice in C57BL/6 background were used. Genotyping of these mice was performed as previously described in detail (Ahmed et al., 2018, Proc Natl Acad Sci USA 115, E8660-E8667). The primer sequences for Neat1 knockout mouse genotyping were followed. Forward primer (5′-3′): GCCATTCAGGCTGCGCAACTG (SEQ ID NO:6); Reverse primer (5′-3′): AGCAGGGATAGCCTGGTCTT (SEQ ID NO:7); Reverse primer (5′-3′): CTAGTGGTGGGGAGGCAGT (SEQ ID NO:8). All procedures complied with the standards for care and use of animal subjects as stated in the Guide for the Care and Use of Laboratory Animals (Institute of Laboratory Resources, National Academy of Sciences, Bethesda, MD).

### Cecal Ligation and Puncture-Induced Sepsis

Cecal ligation and puncture were performed as described previously (Li et al., 2018, The Journal of infectious diseases 218, 1995-2005). Mice were anaesthetized with isoflurane, and a midline incision was made below the diaphragm to expose the cecum. The cecum was ligated at the cecal junction with a 5-0 silk suture without interrupting intestinal continuity and punctured once with a 22-gauge needle. A small amount of stool was extruded through the puncture site. Sham operation was performed in the same way as CLP but without ligation and puncture of the cecum. Mice received the antibiotic imipenem (25 mg/kg, subcutaneously) at 6, 24, and 48 hours after CLP.

### Behavioral Tests

All behavioral tests were performed between 2:00 μm and 5:00 μm during the light cycle in a sound-isolated room. To avoid interference of fecal and urination from other mice, the trial site was thoroughly cleaned with 75% ethanol after each test. Mice were subjected to behavioral tests at least 2 weeks after surgery to allow mice to physically recover from sepsis so that physical weakness will not be considered as a confounder of the measurements. Mice were subjected to an open field test at 2 weeks after CLP and contextual fear conditioning test at 6 weeks after CLP. Behavioral tests were rigorously conducted by an experimenter blinded to the genotype and treatment of the animals.

### Open Field Test (OF)

The open-field test is a widely used model of anxiety-like behavior developed to evaluate emotionality in animals. It involves subjecting an animal to an unknown environment whose escape is prevented by surrounding walls (Savi et al., 2021, Neurosci Biobehav Rev 124, 386-404). Mice were placed in an open field arena (40 cm×40 cm square plexiglass box with walls ˜30 cm tall) in a brightly lit room and allowed to explore freely for 5 minutes. Time spent in center (s), and time spent in the periphery (s) were recorded by live video tracking and EthoVision XT software.

### Contextual Fear Conditioning Test (CFC)

Sepsis-induced cognitive impairment was evaluated by assessing the fear memory processes through re-experiencing symptoms. The measured variable was the time the animal spent freezing, taken as an index of fear in rodents (Savi et al., 2021, Neurosci Biobehav Rev 124, 386-404). An animal was considered to be freezing when crouching, without any visible body movement of the body and head, except for breathing. To evaluate freezing behavior in response to contextual cues, a paradigm using fear conditioning chambers was employed. For this test, mice were singly placed into a plexiglass arena (17 cm×17 cm with foot shock grid floor) within a sound-attenuated chamber (Ugo Basile). Before testing begins, mice were acclimated to the testing room for 30 minutes. On Day 1, mice were singly placed into arenas for 119 s before delivery of a 1 s, 1-mA foot shock. On Day 2 (24 h later), mice were singly placed into the same context and allowed to explore freely for 10 min. Freezing behavior at the last 5 min was recorded using a high-speed digital IR camera and analyzed using EthoVision XT software (Noldus Information Technology). Memory was expressed as the percentage of time the animals spend exhibiting this defensive behavior, and this measure can be used as a retention score; the better the memory, the more time the animal spent in freezing behavior.

RNA Extraction, Reverse Transcription (RT) and Real-Time qPCR

Total RNA was extracted with Trizol (Invitrogen, CA, USA) according to the manufacture's instruction. Then 500 ng RNA was transcripted into cDNA using the High Capacity cDNA Reverse Transcription Kit (Thermo, USA). Quantitative real-time PCR was completed with SYBR PCR Master Mix (QIAGEN, USA) according to manufacturer's protocol. GAPDH was used as the internal control to normalize the mRNA level. Relative mRNA level was calculated by 2^{-ΔΔet} comparative method. Related primer sequences were listed in Table 1.

TABLE 1
Primers used for quantitative RT-qPCR (F: forward; R: reverse)

| Gene Name | Species | Sequence (5′-3′) |
|---|---|---|
| c-fos | Mouse | F: CGGGTTTCAACGCCGACTA (SEQ ID NO: 9) |
| | Mouse | R: TTGGCACTAGAGACGGACAGA (SEQ ID NO: 10) |
| Egr1 | Mouse | F: TATACTGGCCGCTTCTCCCT (SEQ ID NO: 11) |
| | Mouse | R: AGAGGTCGGAGGATTGGTCA (SEQ ID NO: 12) |
| Arc | Mouse | F: AAGTGCCGAGCTGAGATGC (SEQ ID NO: 13) |
| | Mouse | R: CGACCTGTGCAACCCTTTC (SEQ ID NO: 14) |
| Bdnf | Mouse | F: TCATACTTCGGTTGCATGAAGG (SEQ ID NO: 15) |
| | Mouse | R: AGACCTCTCGAACCTGCCC (SEQ ID NO: 16) |
| Homer1 | Mouse | F: CCCTCTCTCATGCTAGTTCAGC (SEQ ID NO: 17) |
| | Mouse | R: GCACAGCGTTTGCTTGACT (SEQ ID NO: 18) |
| Nrn1 | Mouse | F: GCGGTGCAAATAGCTTACCTG (SEQ ID NO: 19) |
| | Mouse | R: CGGTCTTGATGTTCGTCTTGTC (SEQ ID NO: 20) |
| Hbb-b1 | Mouse | F: GCACCTGACTGATGCTGAGAA (SEQ ID NO: 21) |
| | Mouse | R: TTCATCGGCGTTCACCTTTCC (SEQ ID NO: 22) |
| Neat1 | Mouse | F: GCTCTGGGACCTTCGTGACTCT (SEQ ID NO: 23) |
| | Mouse | R: CTGCCTTGGCTTGGAAATGTAA (SEQ ID NO: 24) |
| GAPDH | Mouse | F: GGCAAATTCAACGGCACAGT (SEQ ID NO: 25) |
| | Mouse | R: GGGTCTCGCTCCTGGAAGAT (SEQ ID NO: 26) |
| Malat1 | Mouse | F: GGGAGTGGTCTTAACAGGGAGGAG (SEQ ID NO: 27) |
| | Mouse | R: GTGCCAACAGCATAGCAGTACACG (SEQ ID NO: 28) |
| HOTAIR | Mouse | F: TCCAGATGGAAGGAACTCCAGACA (SEQ ID NO: 29) |
| | Mouse | R: ATAGATGTGCGTGGTCAGATCGCT (SEQ ID NO: 30) |
| PVT1 | Mouse | F: CCTGGATGCCCACTGAAAAC (SEQ ID NO: 31) |
| | Mouse | R: GATAGACTGCTTGCCAGGGG (SEQ ID NO: 32) |
| HIF-2α | Mouse | F: CTGAGGAAGGAGAAATCCCGT (SEQ ID NO: 33) |
| | Mouse | R: TGTGTCCGAAGGAAGCTGATG (SEQ ID NO: 34) |

### Protein Extraction and Western Blotting

Protein lysates were extracted from N2a cells, primary neuronal cells or brain hippocampus tissue. Briefly, after rinsed with PBS, cells were harvested using RIPA buffer (Fisher) with Protease/Phosphatase Inhibitor Cocktail (CST, 5872S) to extract proteins. Following sonication and centrifugation, protein lysate was quantified by BCA assay and loaded onto 10-15% SDS-PAGE gel at 20-40 g per lane. Antibodies used in this study were: β-actin (CST, A5316, Rabbit, 1:1000), Hbb (NOVUS, H00003043-M02, Mouse, 1:500), PSD-95 (Invitrogen, 51-6900, Rabbit, 1:500), Ubiquitin (CST, 43124S, Rabbit, 1:1000). Images were acquired by ChemiDoc™ Touch Imaging System (Bio-Rad) and band densities were quantified using the ImageJ software.

### Mass Spectrometry (MS)

Mice were subjected to sham and CLP and neuronal cells were isolated 24 h after surgery. The protein was precipitated by RNA protein pull-down assay (Pierce magnetic RNA-Protein pull-down kit, Thermo Scientific, USA) in lysed neuronal cells and subjected to specific digestion with trypsin. The peptides were subsequently analyzed using LC-MS/MS with an EASY nLC 1200 in-line with the Orbitrap Fusion Lumos Tribrid Mass Spectrometer (Thermo Scientific) with instrument control software v. 4.2.28.14 (scanning range was 375-1500 m/z). For protein identification and label-free quantification the LC-MS/MS data were searched using the MaxQuant v.1.6.10 against a mouse SwissProt reviewed database with 17,034 proteins and a database of common contaminants. Common contaminants, reversed database hits, and proteins identified by one modified peptide were removed and the LFQ normalized protein intensities were log 2 transformed.

### RNA-Protein Pull-Down Assay

RNA-protein pull-down assays were carried out using the Pierce Magnetic RNA-Protein pull-down kit essentially following the protocol provided by the manufacturer (Thermo Scientific). A pBLUNT vector containing full-length mouse Neat1 V1 sequence was utilized to synthesize sense or antisense probes in vitro by using T3 or T7 promoter, respectively. Subsequently the RNA probes were labeled with biotinylated cytidine bisphosphate and captured by streptavidin magnetic beads. Proteins were extracted from N2a cell protein and then incubated with the biotin-labeled sense or antisense Neat1 probe coupled to the streptavidin magnetic beads. The RNA-bound proteins were eluted for MS or Western blot analysis as described above.

### RNA Immunoprecipitation (RIP)

RIP was performed using the Magna RIP™ RNA-Binding Protein Immunoprecipitation Kit according to the manufacturer's instructions (Millipore, USA). Briefly, N2a cells were lysed and incubated with Hbb antibody (NOVUS, H00003043-M02, Mouse, 10 g) or control IgG (10 g) conjugated with magnetic beads 50 μL (Dynabeads Protein G, Invitrogen) overnight. The beads, protein, and mRNA complexes were immunoprecipitated and then magnetically separated. The mRNAs were purified and were quantified by RT-qPCR using mouse Neat1 and Malat1 primer listed in Table 1. The RT-qPCR product was also visualized in an agarose gel.

### RNA-Fish/if (Immunofluorescence)

Briefly, seeded cells or sections of brain tissues were fixed with 4% paraformaldehyde and treated with 0.5% Triton for 10 min. The sections were blocked with goat serum (5%, Invitrogen) for 30 minutes. The sections were incubated with anti-NeuN (Abcam, mouse, 1:500) antibodies and stained with secondary antibody (488 nm anti-mouse secondary antibody, 1:250 dilution, Invitrogen) diluted in blocking buffer for 1 hour at room temperature. The sections were fixed with 4% paraformaldehyde for 15 min followed by pre-hybridization. Overnight hybridization was performed with a 10 mM mouse Neat1 probe labeled with Quasar 570 Dye (Biosearch Technologies). RNA-FISH/IF was performed using RNA-FISH kit (Biosearch Technologies) according to the manufacturer's instructions. Sections were immersed with mounting medium (ProLong Gold anti-fade reagent with DAPI, Invitrogen) to visualize nuclei and imaged using confocal microscopy (TCS SP8, Leica). The numbers of Neat1 foci positive cells were counted in 4 random fields from 3 biological samples in each group. To determine Neat1 co-localization with Hbb in N2a cells, The cells were seeded on chamber slides. Subsequently RNA-FISH/IF was carried out as described above using anti-Hbb (Invitrogen, PA5-60287, rabbit, 1:2000) antibody and Neat1 probe.

### GapmeR Delivery

Cells were transfected at 60-80% confluence with 20-50 nmol/L control GapmeR or GapmeR (Qiagen) targeting Neat1 (#1, #2) or Hbb using Lipofectamine 2000 (Invitrogen, Carlsbad, CA, USA) according to manufacturer's instructions. Forty-eight hours after transfection, the cells were harvested for further analysis.

Mice were subjected to CLP and administered intravenously control or Neat1 GapmeR #1 (10 nmol/kg body weight) at 4 h after CLP.

### Cell Culture

Mouse neural crest-derived cell line (Neuro-2a, N2a) were obtained from the American Type Culture Collection (ATCC, Manassas, VA, USA). Cells were grown at 37° C. and in 5% CO₂. Cells were incubated for 16 h in an atmosphere of either normoxia (21% oxygen) or hypoxia (1% oxygen). HIF-2α was suppressed by specific siRNA (75377553, Invitrogen, USA).

Isolation and culture of primary mouse neuron cells: Primary neuronal cultures were prepared from newborn 1-3 days C57BL/6 mouse brains. Neurons were isolated using Neuron isolation kit (Miltenyi Biotec Inc., Auburn, CA). Briefly, non-neuronal cells in single-cell preparations from whole mouse brain digests were depleted by negative selection. Isolated neurons were plated onto poly-D-lysine coated dishes and cultured in neuronal medium (Catalog: 1521, ScienCell Research Laboratories, Carlsbad, CA) supplemented with neuronal growth supplement and 1% penicillin/streptomycin (ScienCell Research Laboratories). Cells were grown at 37° C. and in 5% CO₂.

### Golgi Staining

The FD Rapid Golgi Stain kit (FD NeuroTechnologies) was used to perform Golgi staining following the vendor's protocol (Kassem et al., 2013, Mol Neurobiol 47, 645-661). The brains were then sliced (100 m/slice) using a cryostat, 5-6 sections/mice. Slides were coded before quantitative analysis, and the person analyzing the slide was blind to the code.

Quantitative differences in the number of dendritic spines on individual dendritic branch orders between conditions were determined as previously described (Kassem et al., 2013, Mol Neurobiol 47, 645-661). Golgi-stained neurons were examined, and z-stack images were captured microscopically via bright field imaging, on a Keyence BZ—X800 microscope and processed using the Keyence software package. Dendritic spines on apical pyramidal neurons were analyzed in the CA1 sub-region of the hippocampus (approximately −1.6 to −2.46 mm from Bregma). Spine numbers were counted and estimated by from first- to fifth-order dendrites (Kassem et al., 2013, Mol Neurobiol 47, 645-661). All protrusions were counted as spines if they were in direct contact with the dendritic shaft and were less than 1 m in length and 0.5 m in width. For every mouse, six segments of dendrites were selected per branch order per region of interest.

### Statistical Analysis

Statistical significance was determined by analysis of variance (ANOVA) or Student's t-test using GraphPad Prism software. A value of p<0.05 was considered statistically significant.

### The Experimental Results are Now Described

Septic mice exhibit anxiety, cognitive impairment and decreased dendritic spine density

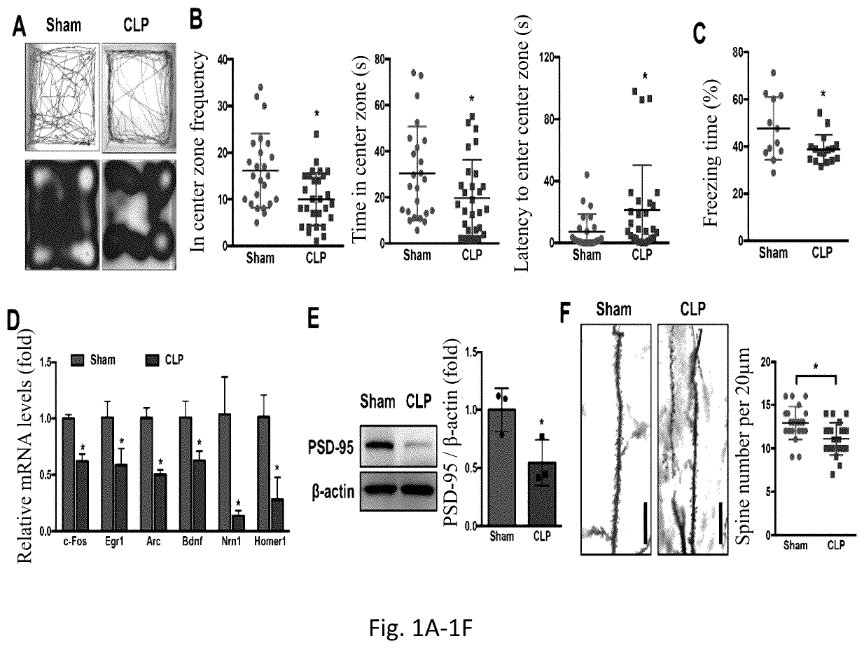

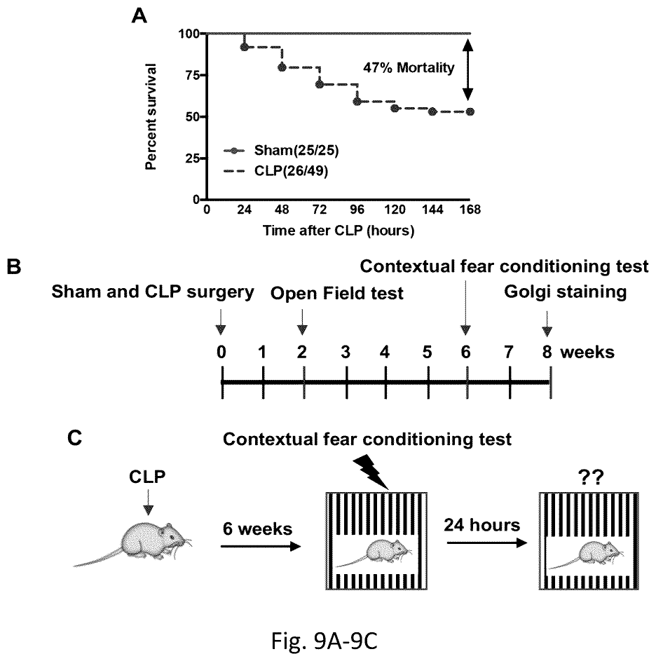

To establish a model of sepsis-associated encephalopathy, sepsis was induced in C57BL/6 mice via cecal ligation and puncture (CLP). A moderate severity CLP model (one puncture with 22G needle) was used, which results in a mortality rate of 47% and the surviving mice were subjected to open field (OF) test for anxiety-like behavior and contextual fear conditioning (CFC) test for hippocampus-dependent memory impairment (FIG. 9A). It was found that septic mice exhibited anxiety-like behavior, as evidenced by visiting the center less frequently, spending less time in the center and taking more time to first enter the center compared with the sham group (p<0.05, FIG. 1A, B and FIG. 9B). In addition, using a CFC test, it was observed that mice had no substantial difference in freezing behavior during the training phase. However, when returned to the testing 24 h later, septic mice exhibited significantly (p<0.05) decreased freezing time compared to sham mice (FIG. 1C and FIG. 9C). These data demonstrated that mice who survive sepsis exhibit anxiety-like behavior and memory impairment.

To further determine whether these behavioral changes were associated with neuronal dysfunction, the expression of immediate early genes (IEGs), which regulate anxiety, memory and neuronal dendritic spine density, was determined. The data showed that the expression of IEGs such as, c-fos, Egr1, Arc, Bdnf Nrn1 and Homer1 were significantly decreased in neuronal cells at 24 h after CLP compared to the sham group (FIG. 1D). Additionally, it was found that the protein levels of PSD-95 (post-synaptic marker) were decreased after CLP compared with the sham group (FIG. 1E). These data suggested that neuronal dysfunction occurs as early as 24 h post sepsis. Neuronal synapses were also stained at 8 weeks after CLP sepsis. Dendritic spine density in the hippocampus was significantly (p<0.05) lower in CLP mice compared to sham mice (FIG. 1F). These data demonstrated that mice surviving sepsis exhibit anxiety-like behavior and memory impairment as well as neuronal dysfunction.

### Neat1 Expression is Increased During Sepsis

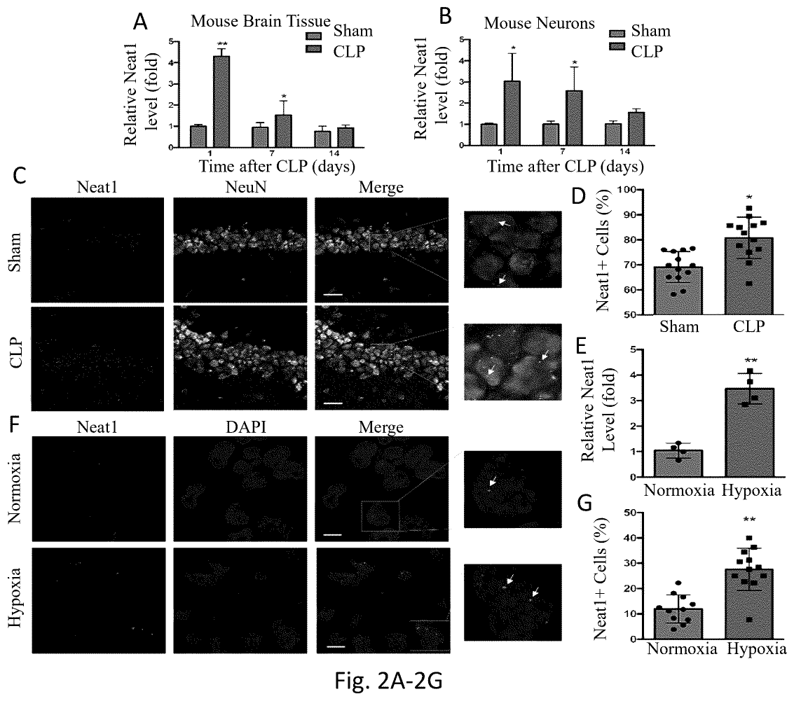

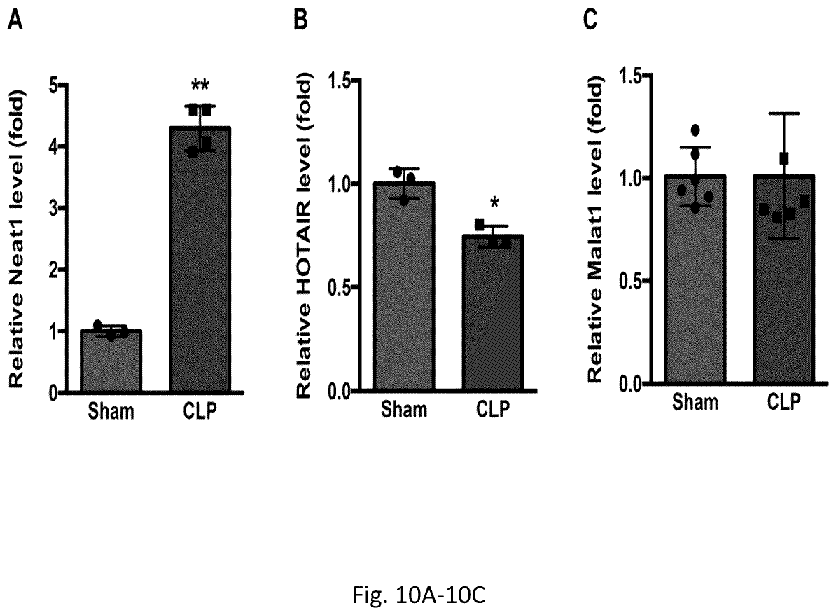

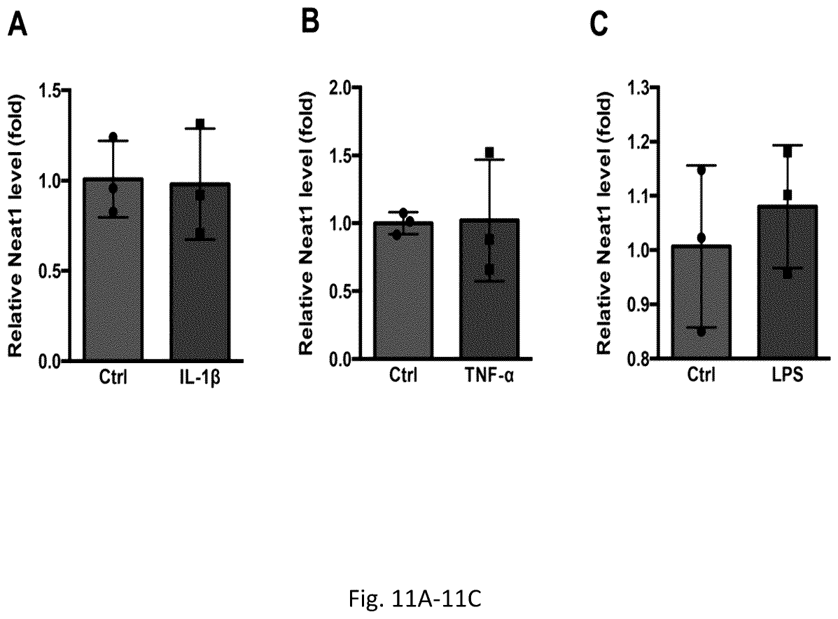

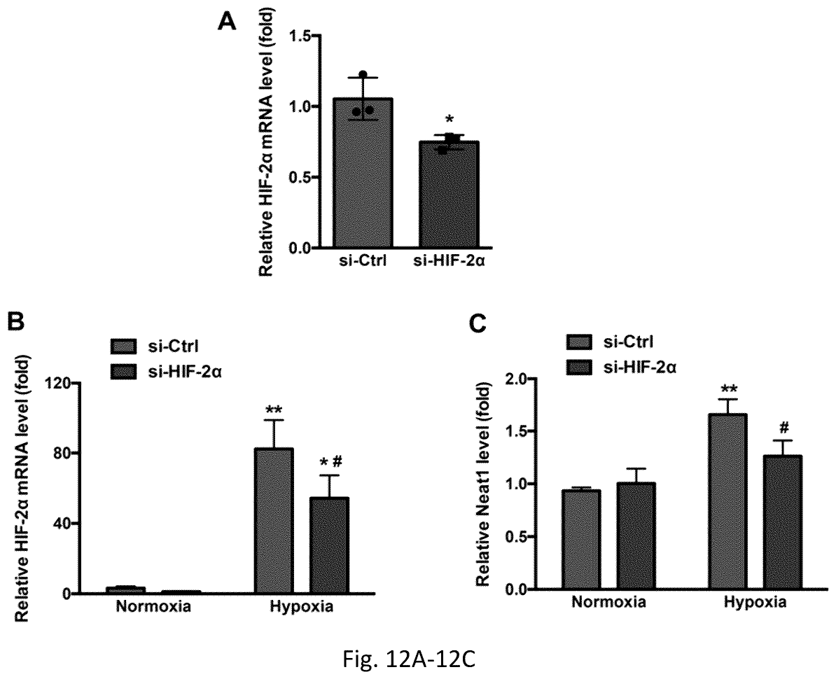

Since previous studies demonstrated that LncRNA play important roles in sepsis and neurodegenerative disease (Butler et al., 2019, Sci Signal 12, (588):eaaw9277; Liu et al., 2019, Eur Rev Med Pharmacol Sci 23, 3933-3939; Riva et al., 2016, Curr Alzheimer Res 13, 1219-1231), sepsis associated LncRNAs (Neat1, HOTAIR, Malat1 and PVT1) were determined in mouse brain tissue at 1 day after CLP. The results showed that only Neat1 levels were increased by 4-fold (FIG. 10A), the other LncRNAs were not significantly changed (FIG. 10B, C). The expression levels of PVT1 were not detectable in brain tissue. Therefore, the Neat1 levels were detected in mouse brain tissues and neurons at 1, 7 and 14 days after CLP. Neat1 levels were significantly (p<0.05) increased in the sepsis group at 1 and 7 days after CLP compared to the sham group and returned to baseline levels at 14 days (FIG. 2A, B). RNA fluorescent in situ hybridization (RNA-FISH) was also performed with a probe specific to the Neat1 variant in the hippocampus region from sham and septic mice at 24 h after CLP. Neurons were stained with NeuN (neuron marker) and Neat1 positive cells were analyzed in all neurons (FIG. 2C). The mean number of Neat1 positive cells were significantly (p<0.05) increased in the septic mice compared to the sham mice although nuclear localization of Neat1 was not affected by sepsis (FIG. 2D). Since hypoxia and inflammation may occur in brain tissue during sepsis (Taccone et al., 2014, Crit Care Med 42, el 14-122; Meneses et al., 2019, Ann N Y Acad Sci 1437, 43-56), Neat1 expression levels were further determined in Neuro-2a (N2a) cells in the condition of hypoxia, or treated with IL-1β, TNF-α, or LPS for 16 h. It was found that N2a cells exposed to hypoxia (1% O₂ levels) exhibited upregulation of Neat1 compared to cells exposed to normoxia (21% O₂ levels) (FIG. 2E) while IL-1β, TNF-α, or LPS does not upregulate Neat1 levels (FIG. 11). RNA-FISH assays further revealed that Neat1 was rarely detectable in the control group (FIG. 2F, upper panel), but was significantly (p<0.01) detectable in the nuclei of N2a cells exposed to hypoxia (FIG. 2F, lower panel and FIG. 2G). In addition, hypoxia can induce Neat1 through HIF-2α-mediated transcriptional activation (Choudhry et al., 2015, Oncogene 34, 4546; Choudhry et al., 2016, Brief Funct Genomics 15, 174−185). N2a cells were treated with siRNA against HIF-2α and the cells were incubated in hypoxic conditions for 16 h (FIG. 12A, B). Treatment with HIF-2α siRNA attenuated hypoxia induced increases of Neat1 (FIG. 12C). These data demonstrated that Neat1 levels were upregulated in sepsis through HIF-2α mediated signaling pathway.

Neat1 Directly Interacts with Hemoglobin Subunit Beta

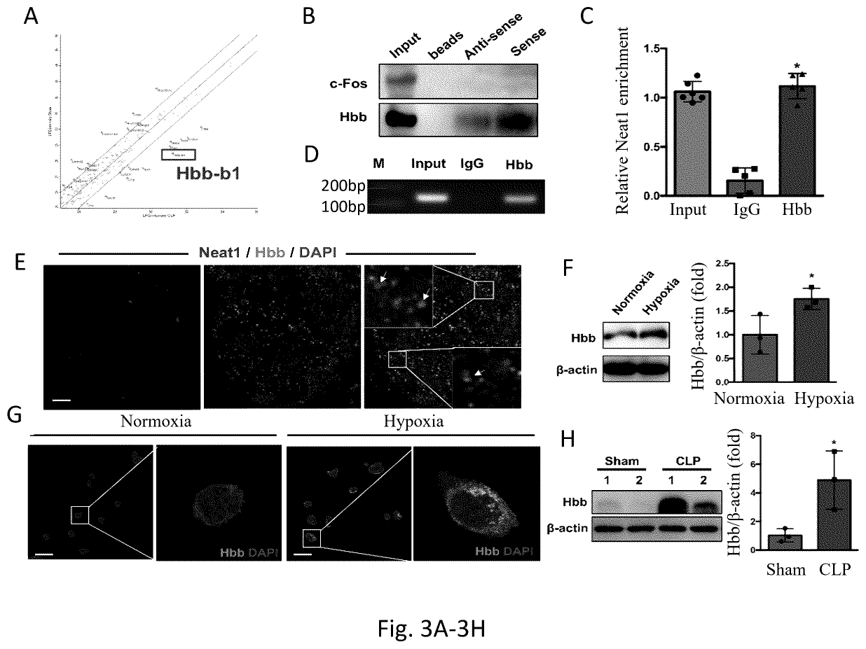

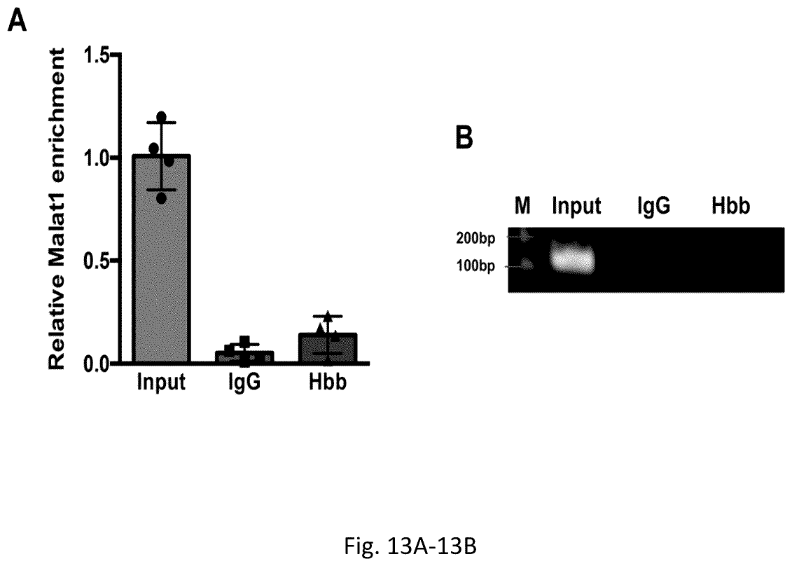

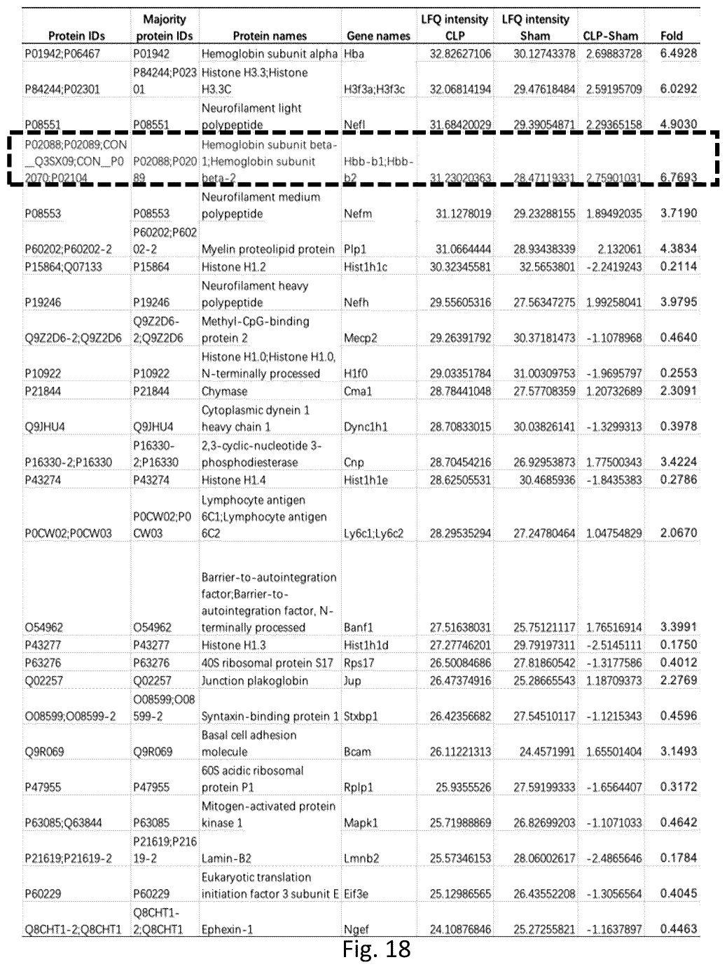

To determine how Neat1 regulates neuronal cell function, proteins binding to Neat1 were first identified using unbiased methods. Brain neuronal cells were obtained from mice at 24 h after sham or CLP. RNA—protein pull-down assays were performed in lysed neuronal cells followed by LC-MS/MS analysis to identify proteins that bind to Neat1 in neurons. Several paraspeckle proteins were identified associated with Neat1. However, those proteins were not significantly altered after sepsis. The proteins that bind to Neat1 and their expression levels were significantly altered (2-fold) in the CLP group compared to the sham group were shown in FIG. 3A. Among these identified proteins, it was found that hemoglobin subunit beta (Hbb) not only bound to Neat1 but also increased most (6.7-fold, p<0.05) in the CLP group compared to the sham group. The details of proteins that bound to Neat1 and their expression levels that were altered in sepsis are listed in FIG. 18. This finding was further validated by repeating an RNA protein pull-down assay in neuronal cell lysate using Neat1 sense and antisense RNA probes and performing western blot against Hbb on the isolated protein, using c-Fos as a negative control. The sense Neat1 probes clearly pulled down Hbb protein, and the antisense probes pulled down a small amount of Hbb while both of them did not pull down the negative control c-Fos (FIG. 3B). These data confirmed the interaction of Neat1 with Hbb. To further confirm the association between Neat1 and Hbb, a RIP assay was used to perform a protein RNA pull-down assay in lysed N2a cells using Malat1 as a negative control. Cell lysates were precipitated with Hbb antibody coupled to protein G beads and pulled down RNA was amplified with Neat1 primers (FIG. 3C) but not Malat1 primers (FIG. 13A) using RT-qPCR. The amplified products were run on an agarose gel. A 150 bp Neat1 PCR product but not Malat1 was observed in the Hbb pull down group (FIG. 3D and FIG. 13B). To further confirm the association of Neat1 and Hbb in neuronal cells, an RNA-FISH/IF assay was performed. RNA-FISH/IF revealed that the colocalization of Neat1 and Hbb in the nucleus of N2a cells (FIG. 3E).

Since Neat1 expression levels were increased by hypoxia, it was determined if Hbb levels were similarly altered. Western blot (FIG. 3F) and Immunofluorescence staining (FIG. 3G) data revealed that Hbb levels were also significantly increased after exposure to hypoxia. Further, it was demonstrated that protein levels of Hbb were increased after CLP compared with the sham group (FIG. 3H). Collectively, these results demonstrated Neat1 directly interacts with Hbb.

### Neat1 Stabilizes Hbb Via Inhibiting Hbb Ubiquitination

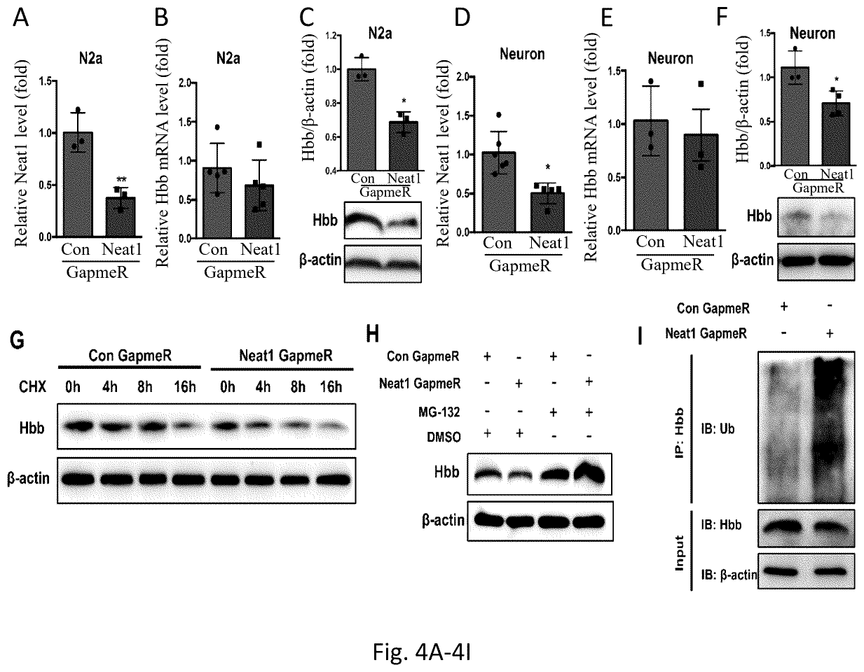

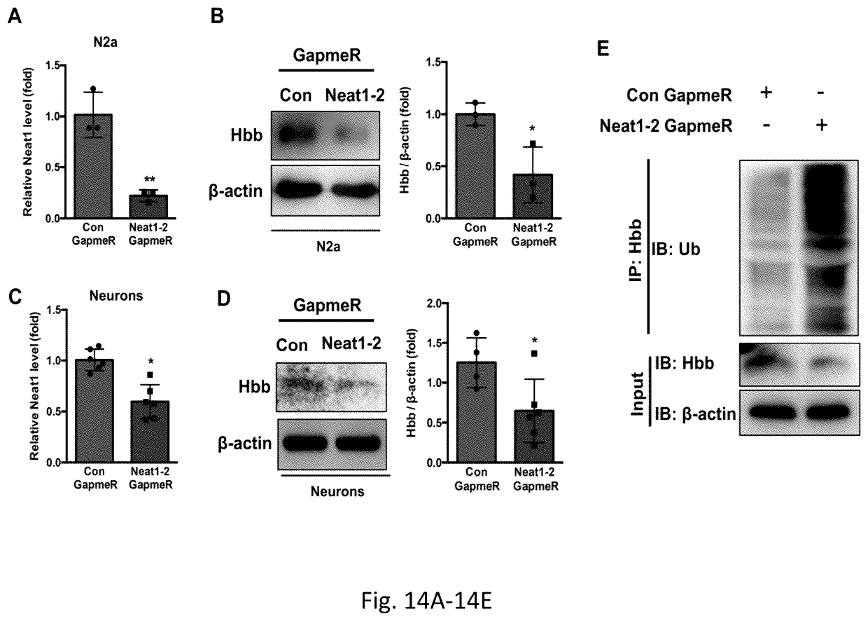

The effects of Neat1 on Hbb expression levels were analyzed using custom designed antisense Neat1 GapmeR based on locked nucleic acids (LNA) technology. N2a cells were transfected with control or Neat1 GapmeR resulting in a significant (p<0.01) decrease in Neat1 levels (FIG. 4A and FIG. 14A). Knockdown of Neat1 in N2a cells did not significantly change the mRNA levels of Hbb; however, the protein levels of Hbb were significantly reduced (FIG. 4B, C and FIG. 14B). These data suggested that Neat1 does not regulate the transcriptional activity of Hbb, but it participates in the regulation of Hbb at the posttranscriptional level. This observation was verified in primary neuron cultures (FIG. 4D, E, F and FIG. 14C, D).

To further determine how Neat1 regulates Hbb expression at the post-transcriptional level, the protein synthesis inhibitor cycloheximide (CHX) was used to block new protein synthesis in N2a cells, which were transfected with control or Neat1 GapmeR. Knock-down of Neat1 significantly (p<0.05) shortened the half-life of Hbb suggesting that Neat1 stabilizes the Hbb protein (FIG. 4G). Further, treatment of control or Neat1 GapmeR transfected N2a cells with the proteasome inhibitor MG-132 reversed Neat1 GapmeR-induced suppression of Hbb levels (FIG. 4H). To further validate that the ubiquitin-proteasome pathway was responsible for the Neat1 knockdown-mediated degradation of Hbb, Co-immunoprecipitation (Co—IP) assays were performed to detect the ubiquitination of Hbb. The lysate of control or Neat1 GapmeR transfected N2a cells were immuno-precipitated with Hbb antibody and immune-blotted with ubiquitin antibody. The ubiquitination of Hbb in N2a cells was significantly (p<0.05) increased by Neat1 knockdown (FIG. 4I and FIG. 14E). Taken together, these results demonstrated that Neat1 directly binds to Hbb and prevents proteasome dependent ubiquitination and degradation of Hbb.

### Neat1/Hbb Axis Suppresses PSD-95 Expression and Dendritic Spine Density

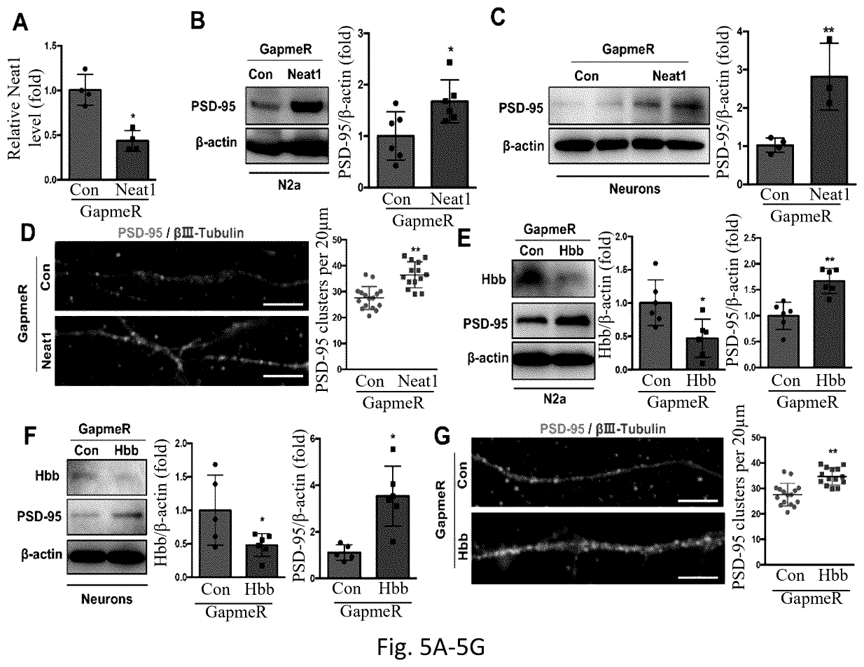

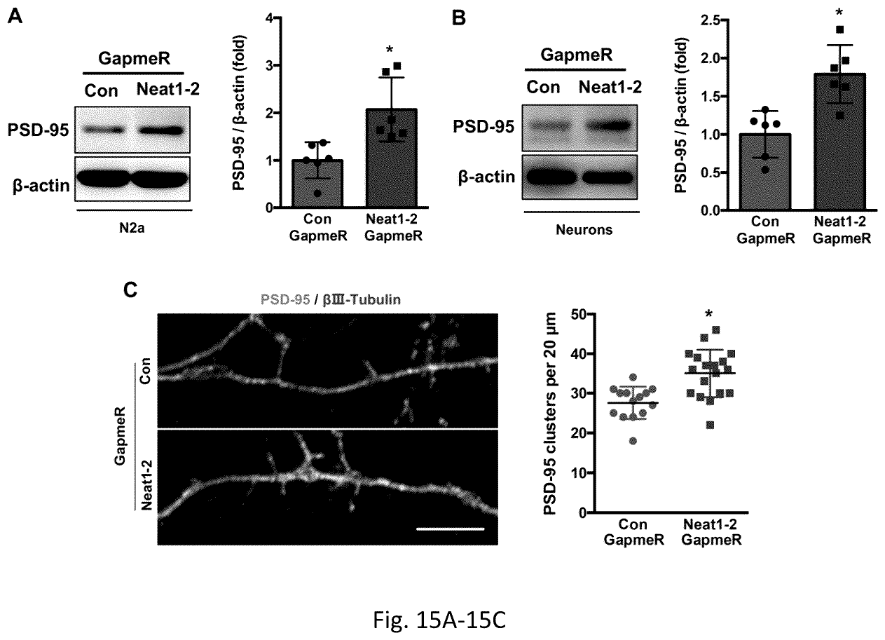

To assess the functional role of Neat1/Hbb axis in cognitive dysfunction in SAE, PSD-95 expression levels were focused on because it plays a critical role in synaptic plasticity and memory (Xu et al., 2016, Curr Opin Neurobiol 21, 306-312). Transfection of Neat1 GapmeR into N2a cells significantly (p<0.05) increased PSD-95 levels (FIG. 5A, B and FIG. 15A). This finding was extended in primary neurons, isolated from the neonatal C57BL/6 mice. These cells were transfected with control or Neat1 GapmeR for 24 h. Neat1 knocked down increased PSD-95 levels (FIG. 5C and FIG. 15B). It was determined if Neat1/Hbb regulates neuronal dendritic spine density by measuring the number of post-synaptic PSD-95 clusters. The cultured primary neurons were transfected with control or Neat1 GapmeR and stained with βIII-tubulin for axons and PSD-95 for dendritic spines. Knockdown of Neat1 significantly (p<0.05) increased dendritic spine density as evidenced by an increased number of PSD-95-positive clusters per 20 m of dendritic section (FIG. 5D and FIG. 15C). To further determine the effect of Hbb on PSD-95 expression and dendritic spine density, Hbb GapmeR was transfected into N2a cells and primary neuronal cells. Hbb GapmeR significantly (p<0.05) decreased Hbb levels and increased PSD-95 levels in N2a cells (FIG. 5E) and primary neuronal cells (FIG. 5F). In addition, knock-down of Hbb also increased dendritic spine density similar to Neat1 knock-down (FIG. 5G). Taken together, these data demonstrated that Neat1/Hbb axis suppresses PSD-95 expression and dendritic spine density in neuronal cells.

### Neat1 Deficiency Attenuates Anxiety and Cognitive Dysfunction Post Sepsis

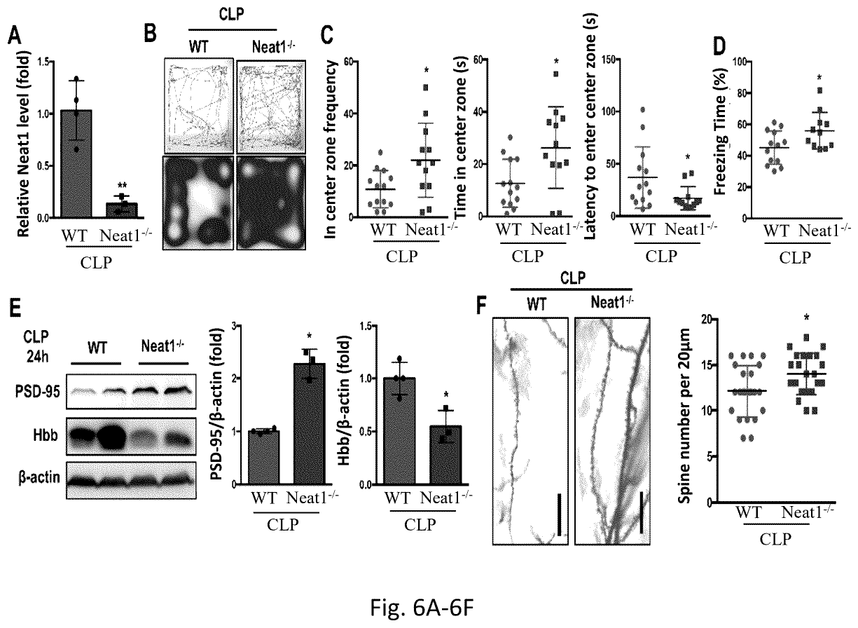

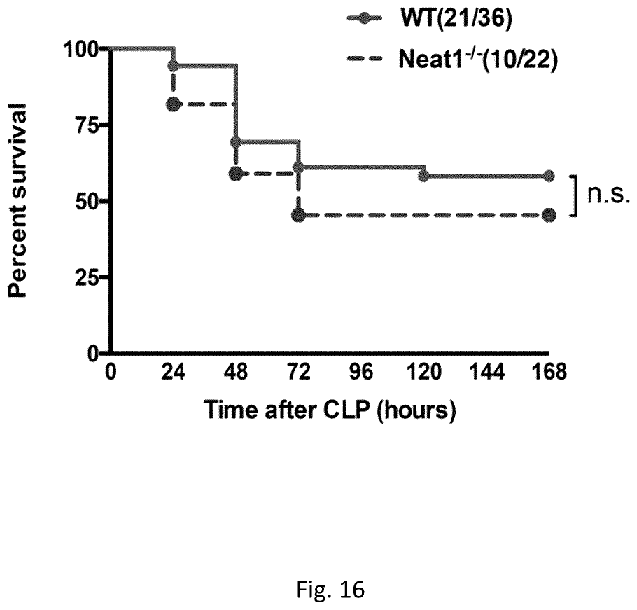

In vitro loss-of-function assays suggested a unique role of the lncRNA Neat1 in promoting postsynaptic-specific gene expression while suppressing synaptic function (FIG. 5). Therefore, next the functional role of Neat1 was determined in synaptic formation in vivo in CLP mice using Neat1^{-/-} mice. Neat1^{-/-} mice do not exhibit apparent gross abnormalities at baseline except for dysfunction of the corpus luteum and mammary gland development in the female KO mice (Nakagawa et al., 2014, Development 141, 4618-4627; Standaert et al., 2014, RNA 20, 1844-1849). No significant difference in mortality was observed between the wild-type and Neat1^{-/-} mice (WT mice: 42%, Neat1^{-/-} mice: 55%) (FIG. 16). It was confirmed that Neat1 expression is completely depleted in the hippocampus of Neat1−/− mice after CLP (FIG. 6A). Neat1^{-/-} mice suppressed post-sepsis anxiety-like behavior, as evidenced by more visiting of the center frequently, spending more time in the center zone and taking less time to first enter the center compared with the wild-type (WT) group (FIG. 6B, C). In addition, using CFC testing, it was observed that Neat1^{-/-} mice exhibited significantly (p<0.05) increased freezing time compared to WT mice (FIG. 6D). The protein levels of Hbb were significantly (p<0.05) decreased while the levels of PSD-95 were significantly (p<0.05) increased in the hippocampus of Neat1^{-/-} mice compared to the WT mice at 24 h after CLP (FIG. 6E). Dendritic spine density in the hippocampus was significantly (p<0.05) increased in Neat1^{-/-} mice compared to WT mice at 8 weeks after CLP (FIG. 6F). Taken together, these data demonstrate that the lncRNA Neat1 plays a critical role in neuronal synaptic function in vitro and in vivo and in post-sepsis neuropsychiatric sequelae.

### Neat1 GapmeR Ameliorates Anxiety and Cognitive Impairment Post Sepsis

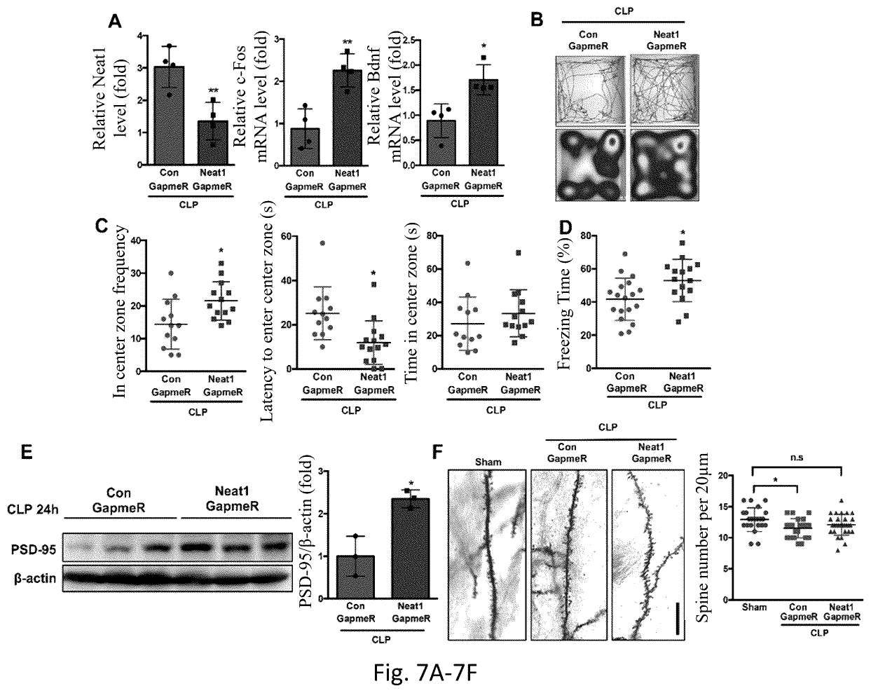

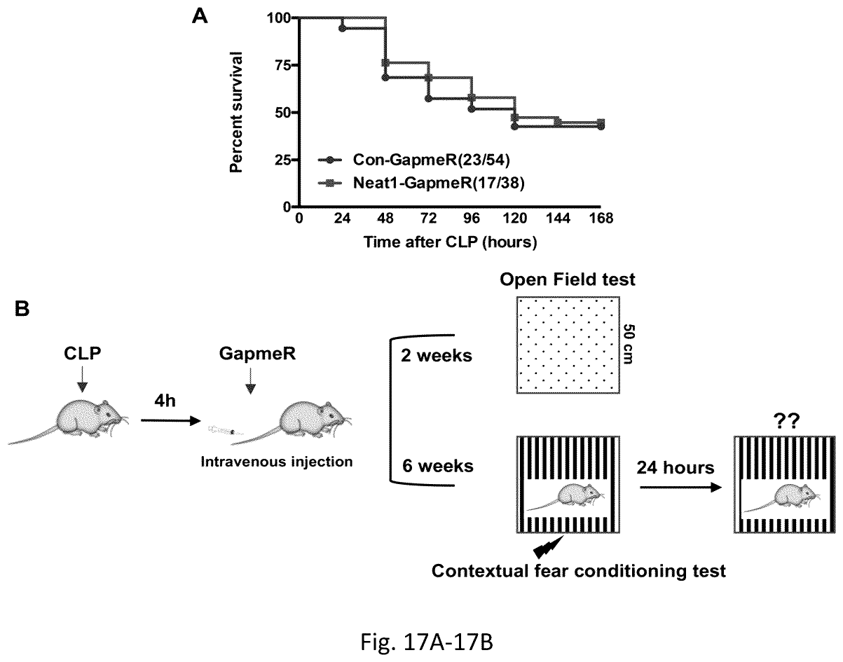

The data suggested that lncRNA Neat1/Hbb axis could be a therapeutic target for SAE. To explore the therapeutic potential of inhibiting the Neat1/Hbb axis, a novel antisense oligonucleotide LNA GapmeR was designed to target Neat1. Previous studies have suggested that under normal physiological conditions, LNA GapmeRs cannot pass across the blood-brain barrier (BBB) with systemic administration (Straarup et al., 2010, Nucleic Acids Res 38, 7100-7111). Since it is known that the BBB is damaged during sepsis (Erikson et al., 2020, Crit Care 24, 385), septic mice were injected intravenously with control or Neat1 GapmeR #1 at 4 h after CLP surgery to determine if Neat1 GapmeR entered the brain after sepsis. Neat1 GapmeR decreased Neat1 levels in brain tissue from septic mice and increased the expression of IEGs (c-Fos and Bdnf) suggesting biologic activity (FIG. 7A). These unexpected data suggest that Neat1 GapmeR take advantage of BBB breakdown during sepsis and enter the brain tissue to exert their biological function. The Neat1 GapmeR injection did not affect mice survival (Control GapmeR: 57%, Neat1 GapmeR: 55%, FIG. 17A). Survivors were subjected to OF and CFC tests at 2 weeks and 6 weeks after CLP (FIG. 17B). Neat1 GapmeR treatment ameliorated CLP sepsis-induced anxiety-like behavior as evidence by visiting the center more frequently and taking less time to first enter the center. The time spent in the center zone was not different between control GapmeR and Neat1 GapmeR treated mice (FIG. 7B, C). Neat1 GapmeR treated mice displayed significantly increased freezing time compared to control GapmeR treated mice (FIG. 7D). At 24 h after CLP, the protein levels of PSD-95 were significantly (p<0.05) increased in brain tissue of mice treated with Neat1 GapmeR compared with the control group (FIG. 7E). Dendritic spine density was determined at 8 weeks after CLP. Treatment with the Neat1 GapmeR ameliorated sepsis-induced dendritic spine loss (FIG. 7F). Taken together these data demonstrated that systemic administration of Neat1 GapmeR ameliorates CLP sepsis-induced anxiety-like behavior, memory impairment and decrease of dendritic spine density.

Despite their frequency and profound impact on the health and well-being of sepsis survivors, little is known about the mechanisms underlying post-septic cognitive and psychiatric sequelae. This knowledge gap has significantly impacted the development of effective preventative and therapeutic strategies to address this major health concern. An important role of the lncRNA Neat1 in the modulation of neuronal synaptic density and neuropsychiatric dysfunction among murine survivors of experimental sepsis has been identified. Specifically, it was shown that CLP-induced sepsis replicated clinical cognitive impairments, including anxiety-like behavior and long-term cognitive deficits in the mouse model, and increased the Neat1 expression in brain tissue, especially in neuronal cells. Furthermore, Neat1 mediated PSD-95 expression by interacting with Hbb protein and regulated dendritic spine density, which is associated with cognition and learning. Deficiency of Neat1 was sufficient to protect post-sepsis cognitive function in Neat1^{-/-} mice. Further, it was found that inhibition of Neat1 using systemic Neat1 GapmeR ameliorated sepsis-related dendritic spine loss and reduced cognitive dysfunction.

Sepsis survivors develop psychiatric sequelae and long-term cognitive impairment following discharge from Intensive care units (ICU), including anxiety, depression, alterations in memory, attention, concentration and/or global cognitive decline, which has a profound impact on their quality of life (Hopkins et al., 2005, Am J Respir Crit Care Med 171, 340-347). The use of behavioral testing in animal models of sepsis is a potentially valuable strategy to understand the onset and the time-course of the mental health problems and cognitive dysfunction associated with sepsis. Open field tests were frequently used for anxiety-like behavior, and contextual fear conditioning tests along with other inhibitory avoidance tests were widely used for assessing hippocampus-dependent memory (Savi et al., 2021, Neurosci Biobehav Rev 124, 386-404). Sepsis-induced by LPS and CLP are the most frequently used models, and both were effective in inducing short- and long-term behavioral impairment (Savi et al., 2021, Neurosci Biobehav Rev 124, 386-404). Compared with the LPS model, the CLP model more closely resembles the progression and characteristics of human sepsis (Dejager et al., 2011, Trends Microbiol 19, 198-208), and it could be a useful tool to study cognitive impairment, anxiety and depression, after sepsis, as well as the mechanisms associated with the recovery from such disorders (Savi et al., 2021, Neurosci Biobehav Rev 124, 386-404). In agreement with previous studies (Denstaedt et al., 2020, Shock 54, 78-86), it was demonstrated that sepsis-surviving mice show persistent impairment since anxiety-like behavior persists for at least 2 weeks after CLP, and memory impairment can be observed at least 6 weeks after sepsis and dendritic spin density remain decreased at 8 weeks after sepsis. These data suggest that the CLP sepsis model may recapitulate human SAE and reinforce the necessity of developing SAE targeted therapy to improve the quality of life for sepsis survivors. In addition, synapses are particularly vulnerable in neurodegenerative diseases and neurological disorders, and LPS decreases synaptic proteins and contributes to the memory deficit (Moraes et al., 2015, Mol Neurobiol 52, 653-663), which is consistent with our data obtained from the CLP model. PSD-95, a major component responsible for synaptic maturation that regulates dendritic spines and developing synapses in the hippocampus, has been recently associated with neuropsychiatric disorders and reduction of PSD-95 was observed in septic mice (Huang et al., 2020, Brain Behav Immun 84, 242-252). The data uncovered a novel mechanism that the Neat1/Hbb axis regulated PSD-95 levels and dendritic spine density in SAE.

Neat1 is an essential component of nuclear paraspeckles (Anantharaman et al., 2016, Sci Rep 6, 34043), which consist of ribonucleoprotein complexes formed around Neat1 (Yamazaki et al., 2018, Mol Cell 70, 1038-1053 e1037). Previous studies investigating Neat1 in the context of epilepsy have reported that, in the excitotoxic conditions of this neurodegenerative disorder, activity-dependent down-regulation of NEAT1 expression is impaired (Barry et al., 2017, Sci Rep 7, 40127). However, observations from studies on other neurodegenerative diseases would suggest that NEAT1 up-regulation is deleterious to neuronal survival (Liu et al., 2018, Clin Exp Pharmacol Physiol 45, 841-848). Moreover, another study found that increased NEAT1 expression might play a notable role in the age-related decline of hippocampus-dependent memory formation (Butler et al., 2019, Sci Signal 12, (588):eaaw9277). Therefore, abnormal Neat1 expression and/or regulation might underlie at least some of the symptoms and phenotypes in common neurological diseases. Interestingly, ample evidence also indicates that Neat1 participates in sepsis-induced organ injury in mice (Chen et al., 2018, Int Immunopharmacol 59, 252-260; Wang et al., 2019, Eur Rev Med Pharmacol Sci 23, 4898-4907; Zhang et al., 2019, Int Immunopharmacol 75, 105731). However, until now, the role of Neat1 in sepsis-associated neuronal dysfunction has been unknown. Since hypoxia may occur in brain tissue during sepsis, a key regulator of the transcriptional responses to hypoxia is hypoxia-inducible factor (HIF) (Kaelin et al., 2008, Mol Cell 30, 393-402). Recently, HIF has been shown to play a critical role in regulating the non-coding transcriptional response (Shih et al., 2017, J Biomed Sci 24, 53). It is reported that hypoxia can induce Neat1 through HIF-2α-mediated transcriptional activation (Choudhry et al., 2015, Oncogene 34, 4546; Choudhry et al., 2016, Brief Funct Genomics 15, 174-185), we found that inhibition of HIF-2α attenuated hypoxia-induced increases of Neat1 in N2a cells. Historically, Neat1 was thought to be exclusive to the nucleus and mainly function as a transcriptional regulator. In this study, it was found that Neat1 was present both in the nucleus and the cytoplasm and exerted post-translational regulation of Hbb via direct binding and inhibition of ubiquitination. Given a report suggesting that Hbb may be a part of a mechanism linking neuronal energetics with epigenetic changes and may function by supporting neuronal metabolism (Brown et al., 2016, J Mol Neurosci 59, 1-17), Hbb was further analyzed. Silencing of Neat1 or Hbb both led to increased PSD-95 and dendritic spines as well as reductions of post-sepsis neuropsychiatric sequelae. The exact role of hemoglobin in neurons is debated although changes in its expression and subcellular localization have been associated with a few neurodegenerative disorders including multiple sclerosis, Alzheimer's disease and Parkinson's disease (Brown et al., 2016, J Mol Neurosci 59, 1-17; Singhal et al., 2018, Mol Neurobiol 55, 8051-8058; Ferrer et al., 2011, J Alzheimers Dis 23, 537-550). This study's observations combined with emerging data suggesting the importance of cell-free hemoglobin in sepsis-related organ failure, provide a strong rationale for ongoing investigation into the role of Hbb in SAE. Previous studies demonstrated that septic mice exhibited decreases of β2-adrenoceptor (β2-AR), increases of neuroinflammation, and decreases of BDNF and PSD-95 expression (Zong et al., 2019, Front Cell Neurosci 13, 293) which are consistent with our findings. The authors demonstrated that treatment with β2-AR agonist clenbuterol ameliorated sepsis-induced cognitive impairment, reduced proinflammatory cytokine and up-regulated BDNF, PSD-95 levels (Zong et al., 2019, Front Cell Neurosci 13, 293). Whether activation of β2-AR could alter Neat1 expression remains to be further investigated.

A comprehensive analysis of the molecular perturbations produced by Neat1 in neuronal cells provides the first evidence of its potential as a therapeutic target in neurons of SAE. To efficiently target Neat1, a novel LNA GapmeR antisense oligonucleotide was used which serves as a unique tool to efficiently knock down lncRNAs (Lennox et al., 2016, Nucleic Acids Res 44, 863-877). LNA GapmeRs have a central DNA gap that binds the RNA target, and triggers its RNase H-dependent degradation; the presence of phosphorothioate confers nuclease resistance in bodily fluids (Stein et al., 2010, Nucleic Acids Res 38, e3), while LNA increases affinity to the target (Roux et al., 2017, Methods Mol Biol 1468, 11-18). LNA GapmeRs are becoming an attractive therapeutic modality to target undruggable pathways in vivo. Prior studies suggest that, in physiological conditions, LNA GapmeRs cannot pass through the blood-brain barrier to reach the brain by systemic administration (Straarup et al., 2010, Nucleic Acids Res 38, 7100-7111). However, this investigation capitalized on the known blood-brain barrier interruption associated with sepsis which allowed intravenously injected Neat1 GapmeRs to enter the brain and impart beneficial effects on both synaptic density and post-sepsis neuropsychiatric symptoms. These findings support the therapeutic potential of LNA GapmeRs in human illness and serve as the basis of ongoing translational studies in SAE.

In summary, the interaction between Neat1 and Hbb was identified, which together regulate dendritic spine density and impact cognitive dysfunctions after sepsis. As a result, Neat1 and Hbb may become a potential target for diagnosis and a treatment strategy for sepsis-associated acute brain dysfunction. Furthermore, these roles may extend to other sources of acute brain dysfunction including COVID-19. Finally, the first evidence of mitigating SAE through a novel antisense oligonucleotide GapmeR based reduction of Neat1 is reported, which could lead to a novel therapeutic strategy to combat SAE.

### Example 2: 1. LncRNA Neat1 Levels are Increased in Patients with Alzheimer's Disease Compared to Controls

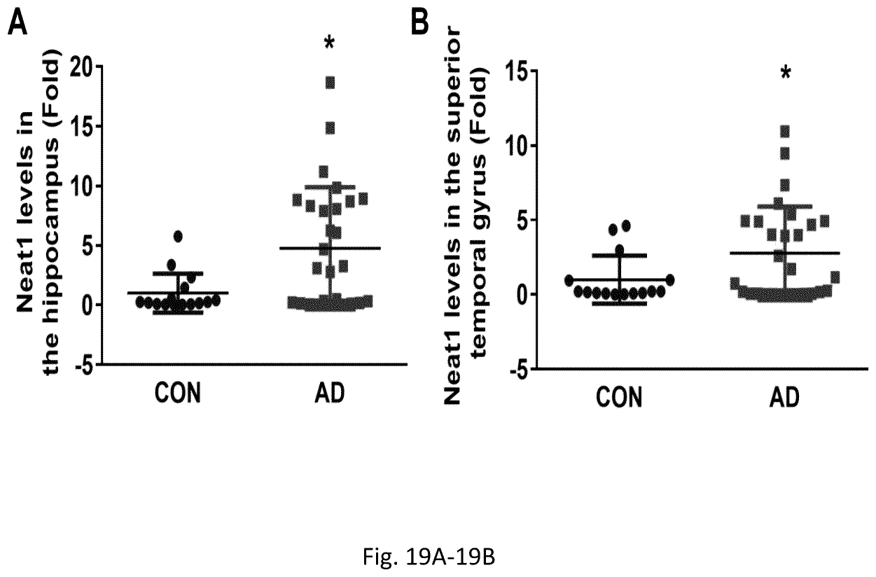

The experiments described below demonstrate that increased Neat1 levels associated with Alzheimer's disease. Postmortem brain tissue of the hippocampus, superior temporal gyrus were obtained from patients with Alzheimer's disease (AD) and controls (CON) from the Brain Bank at MUSC. RNAs were isolated from 100 mg brain tissue using TRIzol reagent, and Neat1 levels were determined by RT-PCR. The data demonstrated that Neat1 levels were significantly higher in the hippocampus and superior temporal gyrus in AD patients compared to controls (FIG. 19). These studies demonstrated that increased Neat1 levels associated with Alzheimer's disease.

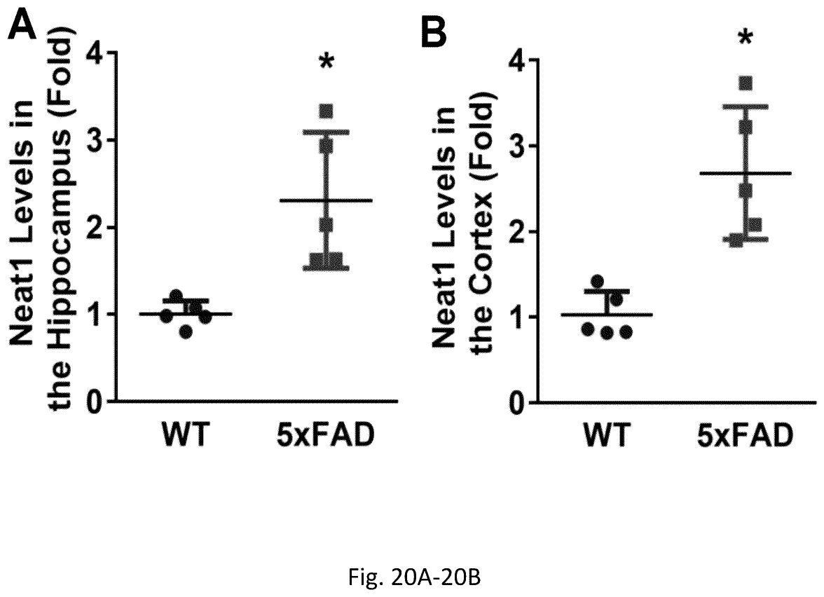

The mouse brain hippocampus and cortex were isolated from WT and 5×FAD mice at 6.5 months of age. Neat1 levels were determined by RT-PCR. It was found that Neat1 levels were significantly increased in the hippocampus and cortex in 5×FAD mice (FIG. 20A, B).

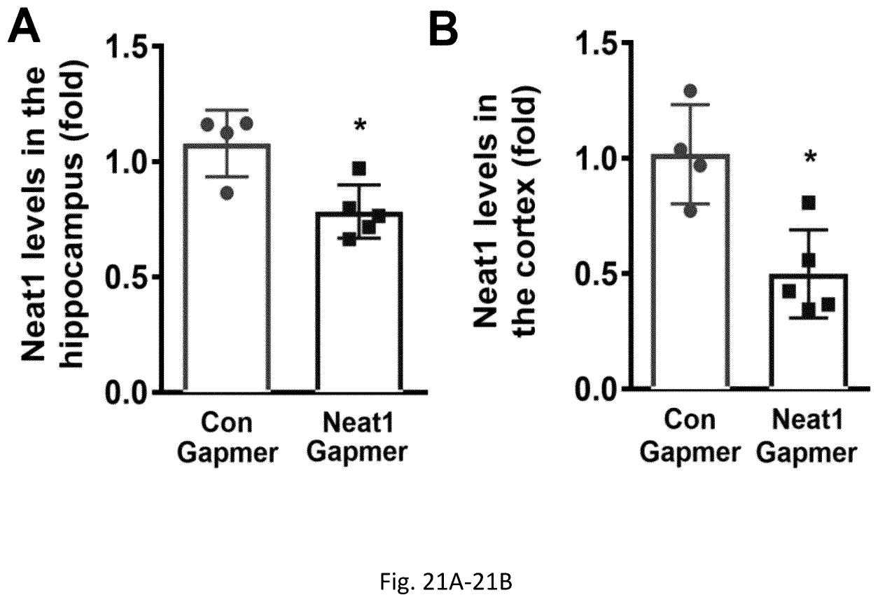

Intrathecal injection of Neat1 Gapmers (5 nmol/kg) effectively suppressed Neat1 levels at 7 days after injection (FIG. 21). Therefore, intrathecal injection of Neat1 Gapmers is one method to potentially treat AD.

### Example 3: Effects of Human Neat1 Gapmers on Neat1 Levels in Human Brain Pericytes

Experiments were performed to determine the effects of human Neat1 GapmeRs in human brain pericytes. 5 human Neat1 Gapmers were designed, wherein each “*”=a phosphorothioated DNA base: G*, A*, T*, C*; and “+”=a LNA modified nucleotide:

Human Gapmer-1
(SEQ ID NO: 1)
5′-+C*+A*+A*G*G*A*A*A*G*T*C*A*T*+C*+G*+C-3′
Human Gapmer-2
(SEQ ID NO: 2)
5′-+A*+A*+T*A*G*A*C*G*T*G*A*G*T*+G*+G*+A-3′
Human Gapmer-3
(SEQ ID NO: 3)
5′-+A*+T*+C*G*A*C*C*A*A*A*C*A*C*+A*+G*+A-3′
Human Gapmer-4
(SEQ ID NO: 4)
5′-+C*+A*+G*G*C*C*G*A*G*C*G*A*A*+A*+A*+T-3′
Human Gapmer-5
(SEQ ID NO: 5)
5′-+A*+C*+G*T*G*A*A*T*A*G*A*C*A*+G*+A*+T-3′

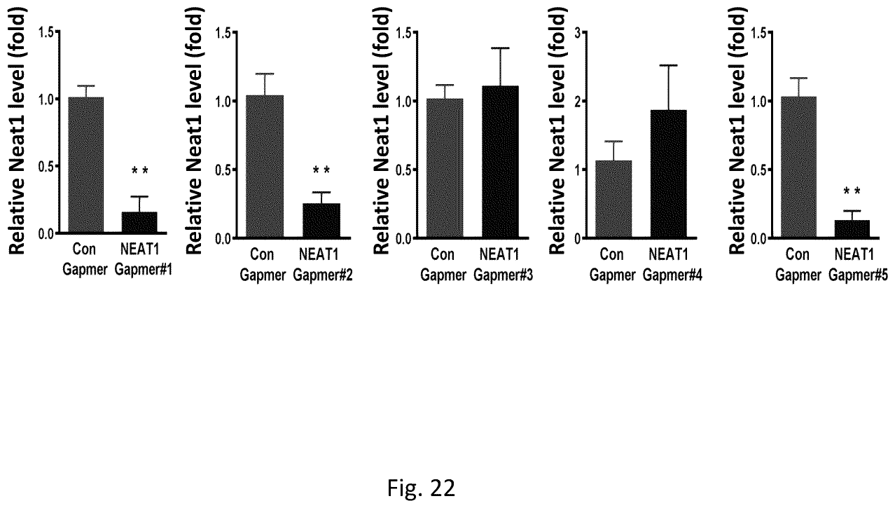

FIG. 22 shows data from human brain pericytes transfected with each Gapmer for 48 hours. Neat1 levels were determined by RT-PCR. **p<0.01, N=5.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A composition for treating or preventing a neurodegenerative disease or disorder, the composition comprising an inhibitor of the level or activity of at least one Neat1 lncRNA molecule or a variant thereof.

2. The composition of claim 1, wherein the inhibitor is at least one selected from the group consisting of a chemical compound, a protein, a peptide, a peptidomemetic, a ribozyme, a small molecule chemical compound, a nucleic acid, a vector, an antibody, an antisense nucleic acid, a GapmeR, an siRNA, an shRNA, and a guide RNA.

3. The composition of claim 2, wherein the composition comprises a GapmeR that targets Neat1 lncRNA.

4. The composition of claim 3, wherein the GapmeR comprises at least one locked nucleic acid (LNA).

5. The composition of claim 4, wherein the GapmeR comprises at least one LNA on each of the 5′ and 3′ end of the GapmeR sequence.

6. The composition of claim 4, wherein the GapmeR comprises at least one phosphorothioated LNA on each of the 5′ and 3′ end of the GapmeR sequence.

7. The composition of claim 3, wherein the composition comprises a GapmeR comprising a nucleotide sequence of SEQ ID NO:1, SEQ ID NO:2, SEQ ID NO:3, SEQ ID NO:4 or SEQ ID NO:5.

8. The composition of claim 1, wherein the modulator inhibits the interaction of Neat1 and Hbb.

9. The composition of claim 1, wherein the disease or disorder is selected from the group consisting of septic induced cognitive impairment, COVID-19 induced cognitive impairment, sepsis-associated encephalopathy (SAE), mild cognitive impairment (MCI), frontotemporal dementia (FTD), epilepsy, traumatic brain injury, schizophrenia, polyQ disorders such as SCA1, SCA2, SCA3, SCA6, SCA7, SCA17, Huntington's disease, Dentatorubral-pallidoluysian atrophy (DRPLA), Alzheimer's disease, Parkinson's disease, amyotrophic lateral sclerosis (ALS), transmissible spongiform encephalopathies (prion disease), tauopathies, and Frontotemporal lobar degeneration (FTLD).

10. A composition for inhibiting the level or activity of at least one Neat1 lncRNA molecule or a variant thereof.

11. The composition of claim 10, wherein the inhibitor is at least one selected from the group consisting of a chemical compound, a protein, a peptide, a peptidomemetic, a ribozyme, a small molecule chemical compound, a nucleic acid, a vector, an antibody, an antisense nucleic acid, a GapmeR, an siRNA, an shRNA, and a guide RNA.

12. The composition of claim 11, wherein the composition comprises a GapmeR that targets Neat1 lncRNA.

13. The composition of claim 12, wherein the GapmeR comprises at least one locked nucleic acid (LNA).

14. The composition of claim 12, wherein the GapmeR comprises at least one LNA on each of the 5′ and 3′ end of the GapmeR sequence.

15. The composition of claim 14, wherein the GapmeR comprises at least one phosphorothioated LNA on each of the 5′ and 3′ end of the GapmeR sequence.

16. The composition of claim 12, wherein the composition comprises a GapmeR comprising a nucleotide sequence of SEQ ID NO:1, SEQ ID NO:2, SEQ ID NO:3, SEQ ID NO:4 or SEQ ID NO:5.

17. The composition of claim 10, wherein the inhibitor inhibits the interaction of Neat1 and Hbb.

18. A method of treating or preventing a neurodegenerative disease or disorder in a subject in need thereof, the method comprising administering to the subject a composition of claim 1.

19. The method of claim 18, wherein the disease or disorder is selected from the group consisting of septic induced cognitive impairment, sepsis-associated encephalopathy (SAE), COVID-19 induced cognitive impairment, mild cognitive impairment (MCI), frontotemporal dementia (FTD), epilepsy, traumatic brain injury, schizophrenia, polyQ disorders such as SCA1, SCA2, SCA3, SCA6, SCA7, SCA17, Huntington's disease, Dentatorubral-pallidoluysian atrophy (DRPLA), Alzheimer's disease, Parkinson's disease, amyotrophic lateral sclerosis (ALS), transmissible spongiform encephalopathies (prion disease), tauopathies, and Frontotemporal lobar degeneration (FTLD).

20. The method of claim 18, wherein the method of administration is intrathecal injection.

21. A method of inhibiting Neat1 expression or activity, the method comprising administering to the subject a composition comprising an inhibitor of claim 10.


SEQUENCE LISTING (34 sequences)

SEQ ID NO: 1 - DNA, 16 bp, synthetic construct
  source 1..16: mol_type = other DNA
  modified_base 1..3: mod_base = OTHER; note = Locked Nucleic Acid
  modified_base 14..16: mod_base = OTHER; note = Locked Nucleic Acid
  modified_base 1..15: mod_base = OTHER; note = phosphorothioated base
caaggaaagtcatcgc

SEQ ID NO: 2 - DNA, 16 bp, synthetic construct
  source 1..16: mol_type = other DNA
  modified_base 1..3: mod_base = OTHER; note = Locked Nucleic Acid
  modified_base 14..16: mod_base = OTHER; note = Locked Nucleic Acid
  modified_base 1..15: mod_base = OTHER; note = phosphorothioated base
aatagacgtgagtgga

SEQ ID NO: 3 - DNA, 16 bp, synthetic construct
  source 1..16: mol_type = other DNA
  modified_base 1..3: mod_base = OTHER; note = Locked Nucleic Acid
  modified_base 14..16: mod_base = OTHER; note = Locked Nucleic Acid
  modified_base 1..15: mod_base = OTHER; note = phosphorothioated base
atcgaccaaacacaga

SEQ ID NO: 4 - DNA, 16 bp, synthetic construct
  source 1..16: mol_type = other DNA
  modified_base 1..3: mod_base = OTHER; note = Locked Nucleic Acid
  modified_base 14..16: mod_base = OTHER; note = Locked Nucleic Acid
  modified_base 1..15: mod_base = OTHER; note = phosphorothioated base
caggccgagcgaaaat

SEQ ID NO: 5 - DNA, 16 bp, synthetic construct
  source 1..16: mol_type = other DNA
  modified_base 1..3: mod_base = OTHER; note = Locked Nucleic Acid
  modified_base 14..16: mod_base = OTHER; note = Locked Nucleic Acid
  modified_base 1..15: mod_base = OTHER; note = phosphorothioated base
acgtgaatagacagat

SEQ ID NO: 6 - DNA, 21 bp, synthetic construct
  source 1..21: mol_type = other DNA
gccattcaggctgcgcaactg

SEQ ID NO: 7 - DNA, 20 bp, synthetic construct
  source 1..20: mol_type = other DNA
agcagggatagcctggtctt

SEQ ID NO: 8 - DNA, 19 bp, synthetic construct
  source 1..19: mol_type = other DNA
ctagtggtggggaggcagt

SEQ ID NO: 9 - DNA, 19 bp, synthetic construct
  source 1..19: mol_type = other DNA
cgggtttcaacgccgacta

SEQ ID NO: 10 - DNA, 21 bp, synthetic construct
  source 1..21: mol_type = other DNA
ttggcactagagacggacaga

SEQ ID NO: 11 - DNA, 20 bp, synthetic construct
  source 1..20: mol_type = other DNA
tatactggccgcttctccct

SEQ ID NO: 12 - DNA, 20 bp, synthetic construct
  source 1..20: mol_type = other DNA
agaggtcggaggattggtca

SEQ ID NO: 13 - DNA, 19 bp, synthetic construct
  source 1..19: mol_type = other DNA
aagtgccgagctgagatgc

SEQ ID NO: 14 - DNA, 19 bp, synthetic construct
  source 1..19: mol_type = other DNA
cgacctgtgcaaccctttc

SEQ ID NO: 15 - DNA, 22 bp, synthetic construct
  source 1..22: mol_type = other DNA
tcatacttcggttgcatgaagg

SEQ ID NO: 16 - DNA, 19 bp, synthetic construct
  source 1..19: mol_type = other DNA
agacctctcgaacctgccc

SEQ ID NO: 17 - DNA, 22 bp, synthetic construct
  source 1..22: mol_type = other DNA
ccctctctcatgctagttcagc

SEQ ID NO: 18 - DNA, 19 bp, synthetic construct
  source 1..19: mol_type = other DNA
gcacagcgtttgcttgact

SEQ ID NO: 19 - DNA, 21 bp, synthetic construct
  source 1..21: mol_type = other DNA
gcggtgcaaatagcttacctg

SEQ ID NO: 20 - DNA, 22 bp, synthetic construct
  source 1..22: mol_type = other DNA
cggtcttgatgttcgtcttgtc

SEQ ID NO: 21 - DNA, 21 bp, synthetic construct
  source 1..21: mol_type = other DNA
gcacctgactgatgctgagaa

SEQ ID NO: 22 - DNA, 21 bp, synthetic construct
  source 1..21: mol_type = other DNA
ttcatcggcgttcacctttcc

SEQ ID NO: 23 - DNA, 22 bp, synthetic construct
  source 1..22: mol_type = other DNA
gctctgggaccttcgtgactct

SEQ ID NO: 24 - DNA, 22 bp, synthetic construct
  source 1..22: mol_type = other DNA
ctgccttggcttggaaatgtaa

SEQ ID NO: 25 - DNA, 20 bp, synthetic construct
  source 1..20: mol_type = other DNA
ggcaaattcaacggcacagt

SEQ ID NO: 26 - DNA, 20 bp, synthetic construct
  source 1..20: mol_type = other DNA
gggtctcgctcctggaagat

SEQ ID NO: 27 - DNA, 24 bp, synthetic construct
  source 1..24: mol_type = other DNA
gggagtggtcttaacagggaggag

SEQ ID NO: 28 - DNA, 24 bp, synthetic construct
  source 1..24: mol_type = other DNA
gtgccaacagcatagcagtacacg

SEQ ID NO: 29 - DNA, 24 bp, synthetic construct
  source 1..24: mol_type = other DNA
tccagatggaaggaactccagaca

SEQ ID NO: 30 - DNA, 24 bp, synthetic construct
  source 1..24: mol_type = other DNA
atagatgtgcgtggtcagatcgct

SEQ ID NO: 31 - DNA, 20 bp, synthetic construct
  source 1..20: mol_type = other DNA
cctggatgcccactgaaaac

SEQ ID NO: 32 - DNA, 20 bp, synthetic construct
  source 1..20: mol_type = other DNA
gatagactgcttgccagggg

SEQ ID NO: 33 - DNA, 21 bp, synthetic construct
  source 1..21: mol_type = other DNA
ctgaggaaggagaaatcccgt

SEQ ID NO: 34 - DNA, 21 bp, synthetic construct
  source 1..21: mol_type = other DNA
tgtgtccgaaggaagctgatg